(19) United States
(12) Patent Application Publication
(10) Pub. No.: US 2026/0177911 A1
(43) Pub. Date: Jun. 25, 2026

(54) ORGANIC SALT, RESIST COMPOSITION INCLUDING THE SAME, AND PATTERN FORMATION METHOD USING THE RESIST COMPOSITION

(71) Applicant: Samsung Electronics Co., Ltd., Suwon-si (KR)

(72) Inventors: Aram JEON, Suwon-si (KR); Minsang KIM, Suwon-si (KR); Kyuhyun IM, Suwon-si (KR); Kyuhwan KIM, Suwon-si (KR); Sunghwan PARK, Suwon-si (KR); Jungkyun OH, Suwon-si (KR); Won Joon CHO, Suwon-si (KR)

(73) Assignee: Samsung Electronics Co., Ltd., Suwon-si (KR)

(21) Appl. No.: 19/216,066

(22) Filed: May 22, 2025

(30) Foreign Application Priority Data
Dec. 19, 2024 (KR) 10-2024-0191730

(51) Int. Cl. G03F 7/004; C07C 323/66; C08F 212/14; C08F 220/18; G03F 7/00; G03F 7/039

(52) U.S. Cl. CPC G03F 7/0042; C07C 323/66; C08F 212/22; C08F 220/1812; G03F 7/0046; G03F 7/0397; C07C 2602/42; C07C 2603/74; G03F 7/70033

(57) ABSTRACT

Provided are an organic salt represented by Formula 1, a resist composition including the same, and a pattern formation method using the resist composition:

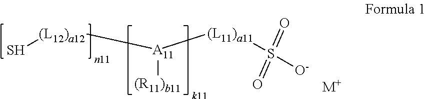

For descriptions of A₁₁, k11, L₁₁, L₁₂, A₁₁, a12, R₁₁, R₁₂, b11, n11 and M⁺ in Formula 1, reference should be made to the specification.

### CROSS-REFERENCE TO RELATED APPLICATION

This application is based on and claims priority under 35 USC § 119 to Korean Patent Application No. 10-2024-0191730, filed on Dec. 19, 2024, in the Korean Intellectual Property Office, the disclosure of which is incorporated by reference herein in its entirety.

### BACKGROUND

### 1. Field

The disclosure relates to an organic salt, a resist composition including the same, and/or a pattern formation method using the resist composition.

### 2. Description of the Related Art

In semiconductor manufacturing, resists of which physical properties change in response to light are being used to form fine patterns. Among these resists, chemically amplified resists have been widely used. In the case of chemically amplified resists, an acid formed through a reaction between light and a photoacid generator reacts with a base resin again to change the solubility of the base resin with respect to a developer, thereby enabling patterning.

In particular, when using high-energy rays with relatively very high energy, such as extreme ultraviolet (EUV) light, the number of photons may be significantly smaller even when light of the same energy is irradiated. Accordingly, there may be a need for resist compositions that may be used more effectively at lower dosages and may provide improved sensitivity, improved resolution and/or reduced defects.

### SUMMARY

Provided are an organic salt capable of providing improved sensitivity, improved resolution and/or reduced defects, a resist composition including the same, and/or a pattern formation method using the resist composition.

Additional aspects will be set forth in part in the description which follows and, in part, will be apparent from the description, or may be learned by practice of the presented embodiments of the disclosure.

According to an embodiment of the disclosure, an organic salt may be represented by Formula 1:

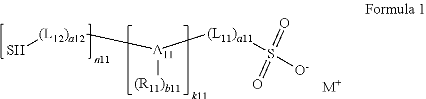

wherein in Formula 1,
A₁₁ is a linear or cyclic C₁-C₃₀ hydrocarbon group optionally including a heteroatom,
k11 is selected from integers of 1 to 4,
L₁₁ and L₁₂ are each independently a single bond, O, S, C(═O), C(═O)O, OC(═O), C(═O)NR₁₂, NR₁₂C(═O), S(═O), S(═O)₂, S(═O)₂O, OS(═O)₂, or a linear, branched or cyclic C₁-C₃₀ divalent hydrocarbon group optionally including a heteroatom,
a11 and a12 are each independently selected from integers of 0 to 4,
R₁₁ and R₁₂ are each independently hydrogen, deuterium, a halogen atom, a cyano group, a hydroxyl group, or a linear, branched or cyclic C₁-C₂₀ monovalent hydrocarbon group optionally including a heteroatom,
b11 is selected from integers of 0 to 10,
any two adjacent groups among R₁₁, L₁₁ and L₁₂ may optionally be bonded to each other to form a ring,
any two adjacent groups among a plurality of R₁₁ groups may optionally be bonded to each other to form a ring,
n11 is selected from integers of 1 to 5, and
M⁺ is a counter cation.

According to an embodiment of the disclosure, a resist composition may include the above-described organic salt, a photoacid generator and a base resin.

According to an embodiment of the disclosure, a method of forming a pattern includes applying the above-described resist composition onto a substrate to form a resist film, exposing at least a portion of the resist film with high-energy rays to form an exposed resist film, and developing the exposed resist film using a developer.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a flowchart illustrating a method of forming a pattern, according to an embodiment;

FIGS. 2A to 2C are cross-sectional side views showing a method of forming a pattern, according to an embodiment;

FIGS. 3A to 3E are cross-sectional side views showing a method of forming a patterning structure, according to an embodiment; and

FIGS. 4A to 4E are cross-sectional side views showing a method of forming a semiconductor device, according to an embodiment.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects. As used herein, the term “and/or” includes any and all combinations of one or more of the associated listed items. Expressions such as “at least one of,” when preceding a list of elements, modify the entire list of elements and do not modify the individual elements of the list. For example, “at least one of A, B, and C,” and similar language (e.g., “at least one selected from the group consisting of A, B, and C” and “at least one of A, B, or C”) may be construed as A only, B only, C only, or any combination of two or more of A, B, and C, such as, for instance, ABC, AB, BC, and AC.

When the terms “about” or “substantially” are used in this specification in connection with a numerical value, it is intended that the associated numerical value includes a manufacturing or operational tolerance (e.g., ±10%) around the stated numerical value. Moreover, when the words “generally” and “substantially” are used in connection with geometric shapes, it is intended that precision of the geometric shape is not required but that latitude for the shape is within the scope of the disclosure. Further, regardless of whether numerical values or shapes are modified as “about” or “substantially,” it will be understood that these values and shapes should be construed as including a manufacturing or operational tolerance (e.g., ±10%) around the stated numerical values or shapes. When ranges are specified, the range includes all values therebetween such as increments of 0.1%.

As the disclosure allows for various changes and numerous embodiments, particular embodiments will be illustrated in the drawings and described in detail in the written description. However, this is not intended to limit the disclosure to particular modes of practice, and it is to be appreciated that all modifications, equivalents, and substitutes that do not depart from the spirit and technical scope of the disclosure are encompassed in the disclosure. In describing the disclosure, when it is determined that the specific description of the related art unnecessarily obscures the gist of the disclosure, the detailed description thereof will be omitted.

Although the terms “first”, “second”, “third”, and the like may be used herein to describe various elements, these terms are only used to distinguish one element from another and the order, type, or the like of the elements are not limited thereby.

A portion of a layer, film, region, plate, or the like described as being “on” or “above” another portion as used herein, it may include not only the meaning of immediately on/under/to the left/to the right in a contact manner, but also the meaning of on/under/to the left/to the right in a non-contact manner.

An expression used in the singular encompasses the expression of the plural, unless it has a clearly different meaning in the context. Unless explicitly described to the contrary, it is to be understood that the terms such as “including” and “having” are intended to indicate the existence of the features, numbers, steps, actions, components, parts, ingredients, materials, or combinations thereof disclosed in the specification and are not intended to preclude the possibility that one or more other features, numbers, steps, actions, components, parts, ingredients, materials, or combinations thereof may exist or may be added.

Whenever a range of values is recited, the range includes all values that fall within the range as if expressly written, and the range further includes the boundaries of the range. Thus, a range of “X to Y” includes all values between X and Y and also includes X and Y.

The expression “Cₓ-C_{y}” used herein refers to the case where the number of carbon atoms constituting a substituent is in a range of x to y. For example, the expression “C₁-C₆” refers to the case where the number of carbon atoms constituting a substituent is in a range of 1 to 6, and the expression “C₆-C₂₀” refers to the case where the number of carbon atoms constituting a substituent is in a range of 6 to 20.

The term “monovalent hydrocarbon group” as used herein refers to a monovalent residue derived from an organic compound including carbon and hydrogen or a derivative thereof, and specific examples thereof include a linear or branched alkyl group (e.g., a methyl group, an ethyl group, a propyl group, an isopropyl group, a butyl group, an isobutyl group, a sec-butyl group, a tert-butyl group, a pentyl group, a neopentyl group, a hexyl group, a heptyl group, a 2-ethylhexyl group, and a nonyl group); a monovalent saturated cycloaliphatic hydrocarbon group (a cycloalkyl group) (e.g., a cyclopentyl group, a cyclohexyl group, a cyclopentylmethyl group, a cyclopentylethyl group, a cyclopentylbutyl group, a cyclohexylmethyl group, a cyclohexylethyl group, a cyclohexylbutyl group, a 1-adamantyl group, a 2-adamantyl group, a 1-adamantylmethyl group, a norbornyl group, a norbornylmethyl group, a tricyclodecanyl group, a tetracyclododecanyl group, a tetracyclododecanylmethyl group, and a dicyclohexylmethyl group); a monovalent unsaturated aliphatic hydrocarbon group (an alkenyl group or an alkynyl group) (e.g., an allyl group); a monovalent unsaturated cycloaliphatic hydrocarbon group (a cycloalkenyl group) (e.g., 3-cyclohexenyl); an aryl group (e. g., a phenyl group, a 1-naphthyl group, and a 2-naphthyl group); an arylalkyl group (e. g., a benzyl group and a diphenylmethyl group); a heteroatom-including monovalent hydrocarbon group (e.g., a tetrahydrofuranyl group, a methoxymethyl group, an ethoxymethyl group, a methylthiomethyl group, an acetamidemethyl group, a trifluoroethyl group, a (2-methoxyethoxy)methyl group, an acetoxymethyl group, a 2-carboxy-1-cyclohexyl group, a 2-oxopropyl group, a 4-oxo-1-adamantyl group, and a 3-oxocyclohexyl group), or a combination thereof. Additionally, some of hydrogens in these groups may be substituted with a moiety including a heteroatom such as oxygen, sulfur, nitrogen, phosphorous or halogen atoms, or some of carbons in these groups may be replaced by a moiety including a heteroatom such as oxygen, sulfur, nitrogen, or phosphorous, and thus these groups may include a cyano group, a nitro group, a hydroxyl group, a thiol group, an amino group, a carboxylate group, an ether moiety, a thioether moiety, a carbonyl moiety, an ester moiety, a phosphonate moiety, a sulfonate moiety, a carbonate moiety, an amide moiety, a lactone moiety, a sultone moiety, or a carboxylic anhydride moiety.

The term “divalent hydrocarbon group” as used herein is a divalent residue and refers to a system in which any one hydrogen atom of the monovalent hydrocarbon group is replaced by a bonding site with a neighboring atom. The divalent hydrocarbon group may include, for example, a linear or branched alkylene group, a cycloalkylene group, an alkenylene group, an alkynylene group, a cycloalkylene group, an arylene group, a group in which some carbon atoms thereof are replaced with a heteroatom, and the like.

The term “alkyl group” as used herein refers to a linear or branched saturated aliphatic monovalent hydrocarbon group, and examples thereof may include a methyl group, an ethyl group, a propyl group, an isobutyl group, a sec-butyl group, a tert-butyl group, a pentyl group, an iso-amyl group, and a hexyl group. The term “alkylene group” as used herein refers to a linear or branched saturated aliphatic divalent hydrocarbon group, and examples thereof may include a methylene group, an ethylene group, a propylene group, a butylene group, and an isobutylene group.

The term “halogenated alkyl group” as used herein refers to a group in which one or more hydrogen of an alkyl group are substituted with a halogen atom, and examples thereof may include CF₃.

The term “alkoxy group” as used herein refers to a monovalent group represented by Formula —OA₁₀₁, wherein A₁₀₁ is an alkyl group. Specific examples thereof may include a methoxy group, an ethoxy group, an isopropyloxy group, and the like.

The term “alkylthio group” as used herein refers to a monovalent group represented by Formula —SA₁₀₁, wherein A₁₀₁ is an alkyl group.

The term “halogenated alkoxy group” as used herein refers to a group in which one or more hydrogen atoms of an alkoxy group are substituted with a halogen atom, and specific examples thereof may include —OCF₃ and the like.

The term “halogenated alkylthio group” as used herein refers to a group in which one or more hydrogen atoms of an alkylthio group are substituted with a halogen atom, and specific examples thereof may include —SCF₃ and the like.

The term “cycloalkyl group” as used herein refers to a monovalent saturated hydrocarbon cyclic group, and specific examples thereof may include monocyclic groups, such as a cyclopropyl group, a cyclobutyl group, a cyclopentyl group, a cyclohexyl group, and a cycloheptyl group, and polycyclic condensed cyclic groups such as a norbornyl group and an adamantyl group. The term “cycloalkylene group” as used herein refers to a divalent saturated hydrocarbon cyclic group, and specific examples thereof may include a cyclopentylene group, a cyclohexylene group, an adamantylene group, an adamantylmethylene group, a norbornylene group, a norbornylmethylene group, a tricyclodecanylene group, a tetracyclododecanylene group, a tetracyclododecanylmethylene group, a dicyclohexylmethylene group, and the like.

The term “cycloalkoxy group” as used herein refers to a monovalent group represented by Formula —OA₁₀₂, wherein A₁₀₂ is a cycloalkyl group. Specific examples thereof may include a cyclopropoxy group, a cyclobutoxy group, and the like.

The term “cycloalkylthio group” as used herein refers to a monovalent group represented by Formula —SA₁₀₂, where A₁₀₂ is a cycloalkyl group.

The term “heterocycloalkyl group” as used herein refers to a group in which some carbon atoms of the cycloalkyl group are substituted with a moiety including a heteroatom, such as oxygen, sulfur, or nitrogen, and the heterocycloalkyl group may specifically include an ether bond, an ester bond, a sulfonate bond, a carbonate, a lactone ring, a sultone ring, or a carboxylic anhydride moiety. The term “heterocycloalkylene group” as used herein refers to a group in which some carbon atoms of the cycloalkylene group are substituted with a moiety including a heteroatom such as oxygen, sulfur, or nitrogen.

The term “heterocycloalkoxy group” as used herein refers to a monovalent group represented by Formula —OA₁₀₃, wherein A₁₀₃ is a heterocycloalkyl group.

The term “alkenyl group” as used herein refers to a linear or branched unsaturated aliphatic hydrocarbon monovalent group including one or more carbon-carbon double bonds. The term “alkenylene group” as used herein refers to a linear or branched unsaturated aliphatic hydrocarbon divalent group including one or more carbon-carbon double bonds.

The term “alkenyloxy group” as used herein refers to a monovalent group represented by Formula —OA₁₀₄, wherein A₁₀₄ is an alkenyl group.

The term “cycloalkenyl group” as used herein refers to a monovalent unsaturated hydrocarbon cyclic group including at least one carbon-carbon double bond. The term “cycloalkenylene group” as used herein refers to a divalent unsaturated hydrocarbon cyclic group including at least one carbon-carbon double bond.

The term “cycloalkenyloxy group” as used herein refers to a monovalent group represented by Formula of —OA₁₀₅, wherein A₁₀₅ is a cycloalkenyl group.

The term “heterocycloalkenyl group” as used herein refers to a group in which some carbon atoms of the cycloalkenyl group are substituted with a moiety including a heteroatom such as oxygen, sulfur, or nitrogen. The term “heterocycloalkenylene group” as used herein refers to a group in which some carbon atoms of the cycloalkenylene group are substituted with a moiety including a heteroatom such as oxygen, sulfur, or nitrogen.

The term “heterocycloalkenyloxy group” as used herein refers to a monovalent group represented by Formula —OA₁₀₆, wherein A₁₀₆ is a heterocycloalkenyl group.

The term “alkynyl group” as used herein refers to a linear or branched monovalent unsaturated aliphatic hydrocarbon group including one or more carbon-carbon triple bonds.

The term “alkynyloxy group” as used herein refers to a monovalent group represented by Formula —OA₁₀₇, wherein A₁₀₇ is an alkynyl group.

The term “aryl group” as used herein refers to a monovalent group including a carbocyclic aromatic system, and examples thereof include a phenyl group, a naphthyl group, an anthracenyl group, a phenanthrenyl group, a pyrenyl group, and a chrysenyl group.

The term “aryloxy group” as used herein refers to a monovalent group represented by Formula —OA₁₀₈, where A₁₀₈ is an aryl group.

The term “heteroaryl group” as used herein refers to a monovalent group including a heterocyclic aromatic system, and examples thereof include a pyridinyl group, a pyrimidinyl group, and a pyrazinyl group. The term “heteroarylene group” as used herein refers to a divalent group including a heterocyclic aromatic system.

The term “heteroaryloxy group” as used herein refers to a monovalent group represented by Formula —OA₁₀₅, where A₁₀₅ is a heteroaryl group.

The term “substituent” as used herein includes deuterium, a halogen atom, a cyano group, a nitro group, a hydroxyl group, a thiol group, an amino group, a carboxylate group, an ether moiety, a thioether moiety, a carbonyl moiety, an ester moiety, a phosphonate moiety, a sulfonate moiety, a carbonate moiety, an amide moiety, a lactone moiety, a sultone moiety, a carboxylic anhydride moiety, a C₁-C₂₀ alkyl group, a C₁-C₂₀ halogenated alkyl group, a C₁-C₂₀ alkoxy group, a C₁-C₂₀ alkylthio group, a C₁-C₂₀ halogenated alkoxy group, a C₁-C₂₀ halogenated alkylthio group, a C₃-C₂₀ cycloalkyl group, a C₃-C₂₀ cycloalkoxy group, a C₃-C₂₀ cycloalkylthio group, a C₆-C₂₀ aryl group, a C₆-C₂₀ aryloxy group, a C₆-C₂₀ arylthio group, a C₁-C₂₀ heteroaryl group, a C₁-C₂₀ heteroaryloxy group, or a C₁-C₂₀ heteroarylthio group; and
a C₁-C₂₀ alkyl group, a C₁-C₂₀ halogenated alkyl group, a C₁-C₂₀ alkoxy group, a C₁-C₂₀ alkylthio group, a C₁-C₂₀ halogenated alkoxy group, a C₁-C₂₀ halogenated alkylthio group, a C₃-C₂₀ cycloalkyl group, a C₃-C₂₀ cycloalkoxy group, a C₃-C₂₀ cycloalkylthio group, a C₆-C₂₀ aryl group, a C₆-C₂₀ aryloxy group, a C₆-C₂₀ arylthio group, a C₁-C₂₀ heteroaryl group, a C₁-C₂₀ heteroaryloxy group, and C₁-C₂₀ heteroarylthio group, each being substituted with deuterium, a halogen atom, a cyano group, a nitro group, a hydroxyl group, a thiol group, an amino group, a carboxylate group, an ether moiety, a thioether moiety, a carbonyl moiety, an ester moiety, a phosphonate moiety, a sulfonate moiety, a carbonate moiety, an amide moiety, a lactone moiety, a sultone moiety, a carboxylic anhydride moiety, a C₁-C₂₀ alkyl group, a C₁-C₂₀ halogenated alkyl group, a C₁-C₂₀ alkoxy group, a C₁-C₂₀ alkylthio group, a C₁-C₂₀ halogenated alkoxy group, a C₁-C₂₀ halogenated alkylthio group, a C₃-C₂₀ cycloalkyl group, a C₃-C₂₀ cycloalkoxy group, a C₃-C₂₀ cycloalkylthio group, a C₆-C₂₀ aryl group, a C₆-C₂₀ aryloxy group, a C₆-C₂₀ arylthio group, a C₁-C₂₀ heteroaryl group, a C₁-C₂₀ heteroaryloxy group, and a C₁-C₂₀ heteroarylthio group, or a combination thereof; or a combination thereof.

Hereinafter, embodiments will be described in detail with reference to the accompanying drawings, wherein like reference numerals denote the same or substantially the same corresponding components throughout the drawings, and a redundant description thereof will be omitted. In the drawings, thicknesses of various layers and regions are enlarged for clarity. Also, in the drawings, the thicknesses of some layers and regions are exaggerated for convenience of description. Meanwhile, embodiments set forth hereinafter are merely for illustrative purposes, and various changes may be made therein.

### [Organic Salt]

An organic salt according to embodiments is represented by Formula 1:

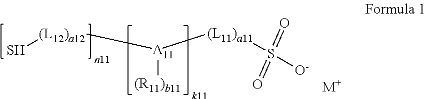

wherein in Formula 1,
A₁₁ is a linear or cyclic C₁-C₃₀ hydrocarbon group optionally including a heteroatom,
k11 is selected from integers of 1 to 4,
L₁₁ and L₁₂ are each independently: a single bond; O; S; C(═O); C(═O)O; OC(═O); C(═O)NR₁₂; NR₁₂C(═O); S(═O); S(═O)₂; S(═O)₂O; OS(═O)₂; or a linear, branched or cyclic C₁-C₃₀ divalent hydrocarbon group optionally including a heteroatom,
a11 and a12 are each independently selected from integers of 0 to 4,
R₁₁ and R₁₂ are each independently hydrogen, deuterium, a halogen atom, a cyano group, a hydroxyl group, or a linear, branched or cyclic C₁-C₂₀ monovalent hydrocarbon group optionally including a heteroatom,
b11 is selected from integers of 0 to 10,
any two adjacent groups among R₁₁, L₁₁ and L₁₂ may optionally be bonded with each other to form a ring,
any two adjacent groups among a plurality of R₁₁ groups may optionally be bonded with each other to form a ring,
n11 is selected from integers of 1 to 5, and
M+ is a counter cation.

For example, in Formula 1, A₁₁ may be a C₁-C₃o alkyl group, a C₂-C₃₀ alkenyl group, a C₃-C₃₀ cycloalkyl group, a C₃-C₃₀ heterocycloalkyl group, a C₃-C₃₀ cycloalkenyl group, a C₃-C₃₀ heterocycloalkenyl group, a C₆-C₃₀ aryl group, or a C₁-C₃o heteroaryl group.

In some embodiments, in Formula 1, A₁₁ may be a methyl group, an ethyl group, an ethenyl group, a cyclopentyl group, a cyclohexyl group, a tetrahydrofuran group, a tetrahydropyran group, a norbornyl group, a norbornenyl group, a norbornadienyl group, a tricyclodecanyl group, a tetracyclododecanyl group, an adamantyl group, an oxa-norbornyl group, an oxa-tricyclodecanyl group, an oxa-tetracyclododecanyl group, an oxa-adamantyl group, a benzene group, a naphthalene group, a phenanthrene group, an anthracene group, a pyrrole group, a furan group, a thiophene group, an indole group, a benzofuran group, a benzothiophene group, a carbazole group, a dibenzofuran group, or a dibenzothiophene group.

In some embodiments, in Formula 1, A₁₁ may be selected from Formulae 9-1 to 9-14:

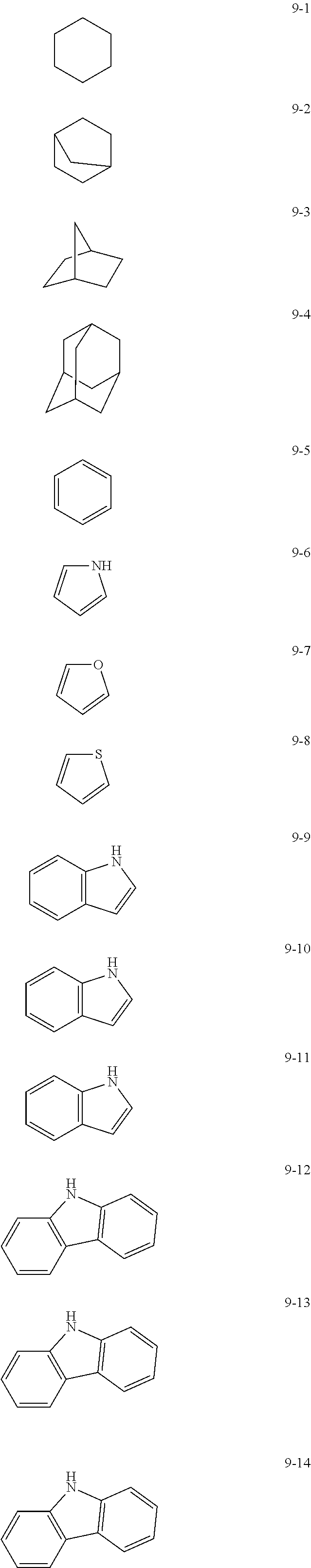

wherein in Formulae 9-1 to 9-14,
n11 hydrogen atoms are *-(L₁₂)ₐ₁₂-SH, and 1 hydrogen atom is *-(L₁₁)ₐ₁₁-SO₃⁻M⁺.

In Formula 1, k11 represents the number of repetitions of A₁₁(R₁₁)_{b11}.

For example, in Formula 1, k11 may be 1 or 2.

For example, in Formula 1, L₁₁ and L₁₂ may each independently be: a single bond; O; C(═O); C(═O)O; OC(═O); C(═O)NH; NHC(═O); a substituted or unsubstituted C₁-C₃₀ alkylene group, a substituted or unsubstituted C₃-C₃₀ cycloalkylene group, a substituted or unsubstituted C₃-C₃₀ heterocycloalkylene group, a substituted or unsubstituted C₂-C₃₀ alkenylene group, a substituted or unsubstituted C₃-C₃₀ cycloalkenylene group, a substituted or unsubstituted C₃-C₃₀ heterocycloalkenylene group, a substituted or unsubstituted C₆-C₃₀ arylene group, a substituted or unsubstituted C₁-C₃₀ heteroarylene group, or a combination thereof.

In some embodiments, in Formula 1, L₁₁ and L₁₂ may each independently be selected from: a single bond; O; C(═O); C(═O)O; OC(═O); and a methylene group, an ethylene group, an n-propylene group, an n-butylene group, an iso-butylene group, a cyclopentylene group, and a cyclohexylene group, each unsubstituted or substituted with deuterium, a halogen atom, a cyano group, a hydroxyl group, an amino group, a carboxylate group, a thiol group, a methyl group, an ethyl group, an n-propyl group, an iso-propyl group, an n-butyl group, an iso-butyl group, a sec-butyl group, a tert-butyl group, a methoxy group, an ethoxy group, a cyclopentyl group, a cyclohexyl group, a phenyl group, or a combination thereof, and a combination thereof.

In some embodiments, in Formula 1, L₁₁ and L₁₂ may each independently be at least one selected from: a single bond; O; C(═O); C(═O)O; OC(═O); and [C(R₉₁)(R₉₂)]ₙ₉₁,
R₉₁ and R₉₂ may each independently be selected from hydrogen, deuterium, a halogen atom, a cyano group, a hydroxyl group, an amino group, a carboxylate group, a C₁-C₂₀ alkyl group, a C₁-C₂₀ halogenated alkyl group, a C₁-C₂₀ alkoxy group, a C₃-C₂₀ cycloalkyl group, a C₃-C₂₀ cycloalkoxy group and C₆-C₂₀ aryl group, and may optionally be bonded to form a ring, and
n91 may be selected from integers of 1 to 3.

Here, n91 represents the number of repetitions of C(R₁₉)(R₉₂).

In an embodiment, R₉₁ and R₉₂ may each independently be selected from hydrogen, deuterium, a halogen atom, a cyano group, a hydroxyl group, a C₁-C₂₀ alkyl group and a C₁-C₂₀ halogenated alkyl group, and n91 may be selected from an integer of 1 to 2.
a11 and a12 represent the number of repetitions of L₁₁ and L₁₂, respectively, and for example, a11 and a12 may each independently be 0, 1, or 2.

For example, in Formula 1, R₁₁ and R₁₂ may each independently be selected from: hydrogen; deuterium; a halogen atom; a cyano group; a hydroxyl group; and a C₁-C₂₀ alkyl group, a C₁-C₂₀ alkoxy group, a C₃-C₂₀ cycloalkyl group, a C₃-C₂₀ cycloalkoxy group, and a C₆-C₂₀ aryl group, each unsubstituted or substituted with deuterium, a halogen atom, a cyano group, a hydroxyl group, an amino group, a carboxylate group, an ester moiety, a sulfonate moiety, a carbonate moiety, a lactone moiety, a sultone moiety, a carboxylic anhydride moiety, a C₁-C₂₀ alkyl group, a C₁-C₂₀ halogenated alkyl group, a C₁-C₂₀ alkoxy group, a C₃-C₂₀ cycloalkyl group, a C₃-C₂₀ cycloalkoxy group, a C₆-C₂₀ aryl group, or a combination thereof.

In some embodiments, in Formula 1, R₁₁ and R₁₂ may each independently be selected from hydrogen; deuterium; a halogen atom; a cyano group; a hydroxyl group; and a C₁-C₂₀ alkyl group, a C₁-C₂₀ cycloalkyl group and a C₆-C₂₀ aryl group, each unsubstituted or substituted with deuterium, a halogen atom, a C₁-C₂₀ alkyl group, a C₁-C₂₀ halogenated alkyl group, a C₃-C₂₀ cycloalkyl group, a C₆-C₂₀ aryl group, or a combination thereof.

In some embodiments, in Formula 1, R₁₁ may be selected from hydrogen, deuterium, a halogen atom, and a cyano group.

In some embodiments, in Formula 1, R₁₂ may be hydrogen or deuterium.

In Formula 1, n11 represents the number of substitutions of *-(L₁₂)ₐ₁₂-SH.

For example, in Formula 1, n11 may be 1 or 2.

For example, in Formula 1, M⁺ may be represented by either of Formulae 2-1 and 2-2:

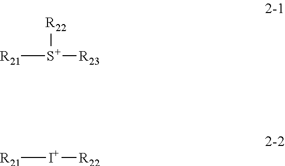

wherein in Formulae 2-1 and 2-2,
R₂₁ to R₂₃ are each independently a linear, branched or cyclic C₁-C₃₀ monovalent hydrocarbon group optionally including a heteroatom, and
any two adjacent groups of R₂₁ to R₂₃ may optionally be bonded with each other to form a ring.

For example, in Formulae 2-1 and 2-2, R₂₁ to R₂₃ may each independently be selected from a C₁-C₂₀ alkyl group, a C₃-C₂₀ cycloalkyl group, and a C₆-C₂₀ aryl group, each unsubstituted or substituted with deuterium, a halogen atom, a cyano group, a hydroxyl group, a carboxyl group, an ester moiety, a sulfonate moiety, a carbonate moiety, a lactone moiety, a sultone moiety, a carboxylic anhydride moiety, a C₁-C₂₀ alkyl group, a C₁-C₃₀ halogenated alkyl group, a C₁-C₂₀ alkoxy group, a C₃-C₂₀ cycloalkyl group, a C₃-C₂₀ cycloalkoxy group, a C₆-C₂₀ aryl group, or a combination thereof.

In some embodiments, in Formulae 2-1 and 2-2, R₂₁ to R₂₃ may each independently be selected from a C₁-C₂₀ alkyl group, a C₃-C₂₀ cycloalkyl group, and a C₆-C₂₀ aryl group, each unsubstituted or substituted with a halogen atom, a cyano group, a hydroxyl group, an ester moiety, a sulfonate moiety, a lactone moiety, a sultone moiety, a carboxylic anhydride moiety, a methyl group, an ethyl group, a propyl group, an isopropyl group, a butyl group, an isobutyl group, a sec-butyl group, a tert-butyl group, a halogenated methyl group, a halogenated ethyl group, a methoxy group, an ethoxy group, a phenyl group, or a combination thereof.

In an embodiment, at least one of R₂₁ to R₂₃ in Formula 2-1 and at least one of R₂₁ and R₂₂ in Formula 2-2 may be a C₆-C₂₀ aryl group substituted with at least one halogen atom.

In an embodiment, R₂₁, R₂₂ or R₂₃ in Formula 2-1 and R₂₁ or R₂₂ in Formula 2-2 may include at least one halogen atom;
R₂₁ or R₂₂ in Formula 2-1 and R₂₁ in Formula 2-2 may include at least one halogen atom, and R₂₃ in Formula 2-1 and R₂₂ in Formula 2-2 do not include a halogen atom; or
R₂₁ to R₂₃ in Formula 2-1 and R₂₁ and R₂₂ in Formula 2-2 may each include at least one halogen atom.

In another embodiment, R₂₁, R₂₂ or R₂₃ in Formula 2-1 and R₂₁ or R₂₂ in Formula 2-2 may include one or two halogen atoms;
R₂₁ or R₂₂ in Formula 2-1 and R₂₁ in Formula 2-2 may each include 1 or 2 halogen atoms, and R₂₃ in Formula 2-1 and R₂₂ in Formula 2-2 do not include halogen atoms; or
R₂₁ to R₂₃ in Formula 2-1 and R₂₁ and R₂₂ in Formula 2-2 may each include one or two halogen atoms.

In some embodiments, in Formula 1, M⁺ may be represented by any one of Formulae 2-11 and 2-12:

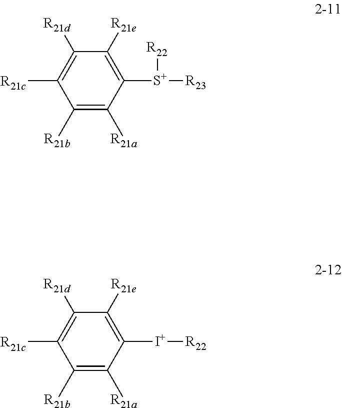

wherein in Formulae 2-11 and 2-12,
R₂₁ₐ to R₂₁ₑ are each independently hydrogen; deuterium; a halogen atom; a cyano group; a hydroxyl group; or a linear, branched or cyclic C₁-C₂₀ monovalent hydrocarbon group optionally including a heteroatom,
R₂₂ to R₂₃ are each independently a linear, branched or cyclic C₁-C₃₀ monovalent hydrocarbon group optionally including a heteroatom, and
any two adjacent groups of R₂₁ₐ to R₂₁ₑ and R₂₂ to R₂₃ may optionally be bonded with each other to form a ring.

In some embodiments, in Formula 1, M⁺ may be represented by any one of Formulae 2-21 to 2-23:

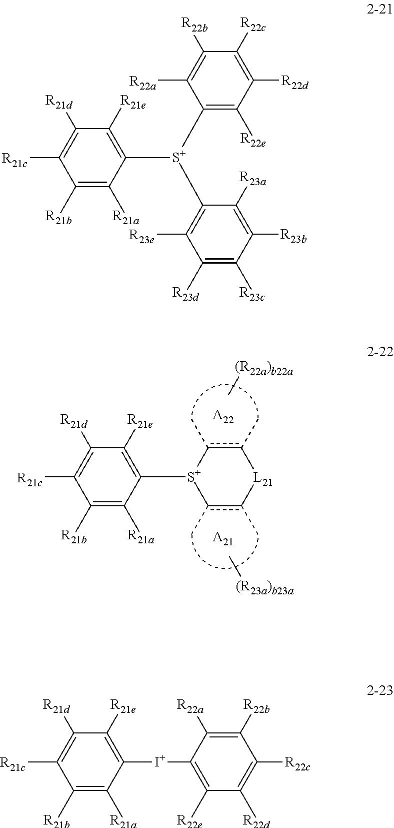

wherein in Formulae 2-21 to 2-23,
R₂₁ₐ to R₂₁ₑ, R₂₂ₐ to R₂₂ₑ and R₂₃ₐ to R₂₃ₑ are each independently hydrogen; deuterium; a halogen atom; a cyano group; a hydroxyl group; or a linear, branched or cyclic C₁-C₂₀ monovalent hydrocarbon group optionally including a heteroatom,
any two adjacent groups of R₂₁ₐ to R₂₁ₑ, R₂₂ₐ to R₂₂ₑ and R₂₃ₐ to R₂₃ₑ may optionally be bonded with each other to form a condensed ring,
b22a and b23a are integers of 1 to 4, respectively,
A₂₁ and A₂₂ are each independently absent or a benzene ring;
[symbol] are each a carbon-carbon single bond or a carbon-carbon double bond;
L₂₁ is a single bond, O, S, CO, SO, SO₂, CRR′, or NR, and
R and R′ are each independently: hydrogen; deuterium; a halogen atom; a cyano group; a hydroxyl group; or a linear, branched or cyclic C₁-C₂₀ monovalent hydrocarbon group optionally including a heteroatom.

In an embodiment, in Formula 1, M⁺ may include at least one halogen.

In an embodiment, in Formula 1, M⁺ may include at least one F or I.

In an embodiment, in Formula 1, M⁺ may include one, two or three I.

In one embodiment, in Formula 1, M⁺ may be selected from Group I:

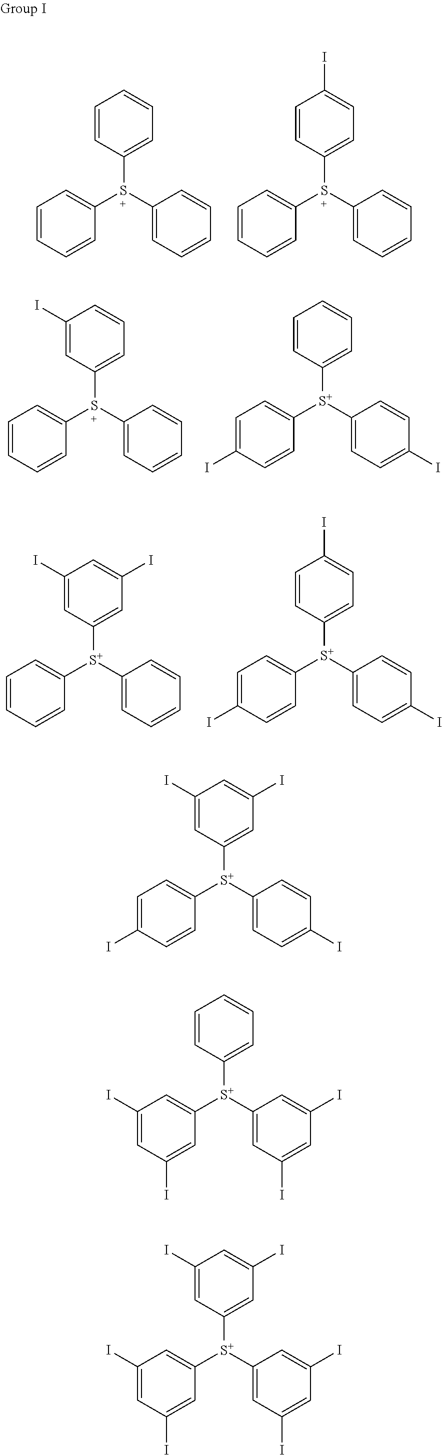

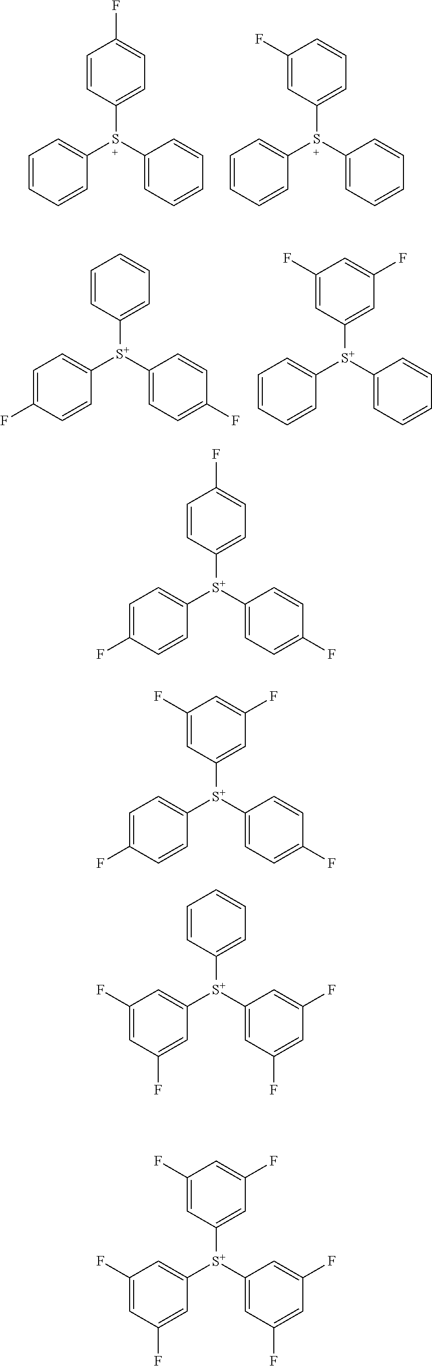

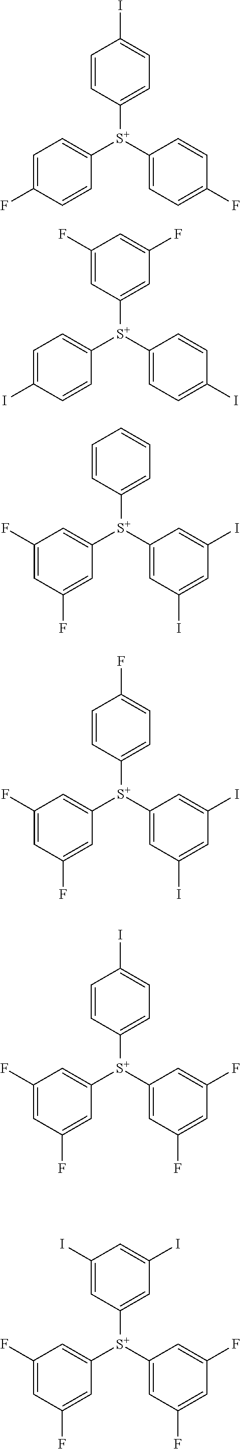 [SMILES: Fc1ccc([S+](c2ccc(F)cc2)c2ccc(I)cc2)cc1 ; Fc1cc(F)cc([S+](c2ccc(I)cc2)c2ccc(I)cc2)c1 ; Fc1cc(F)cc([S+](c2ccccc2)c2cc(I)cc(I)c2)c1 ; Fc1ccc([S+](c2cc(F)cc(F)c2)c2cc(I)cc(I)c2)cc1 ; Fc1cc(F)cc([S+](c2ccc(I)cc2)c2cc(F)cc(F)c2)c1 ; Fc1cc(F)cc([S+](c2cc(F)cc(F)c2)c2cc(I)cc(I)c2)c1]

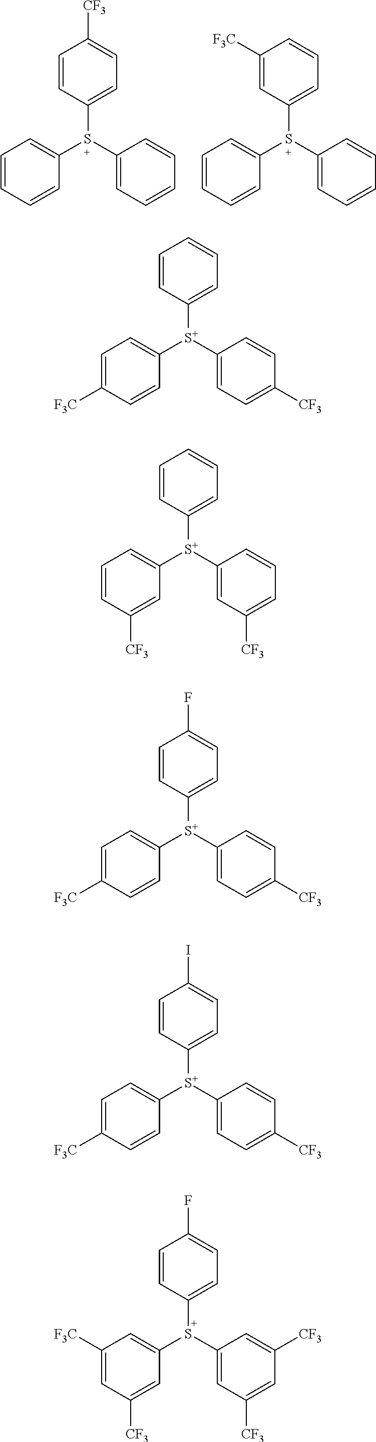

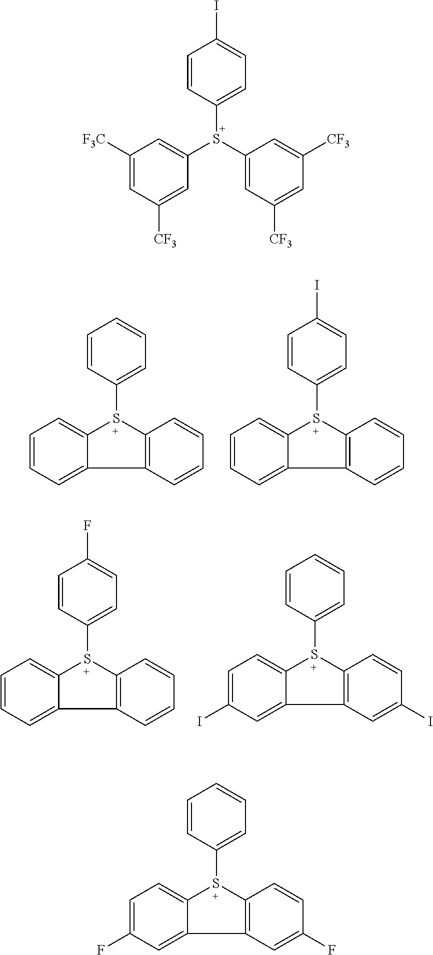

In an embodiment, the organic salt represented by Formula 1 may be selected from Group II:

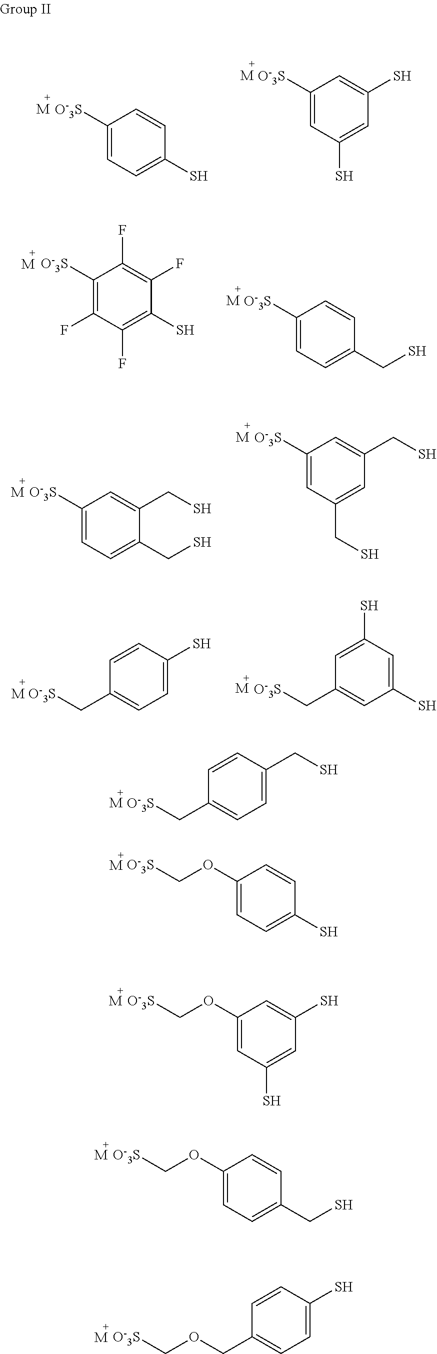

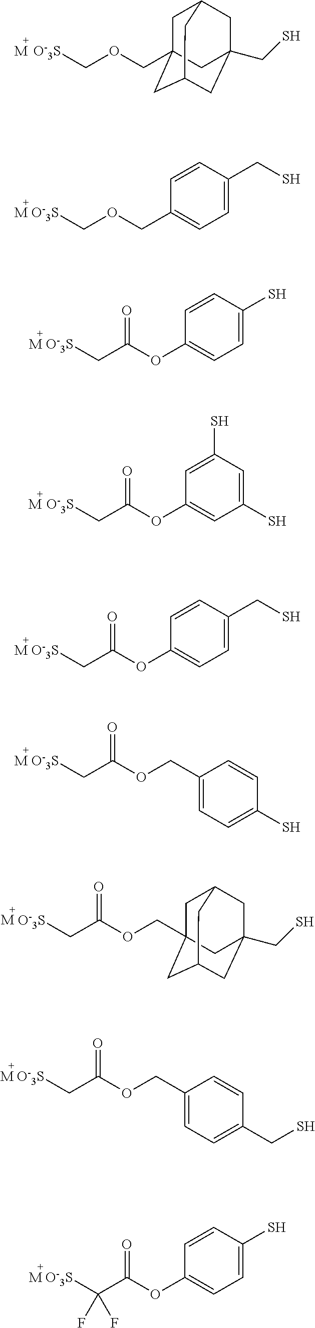

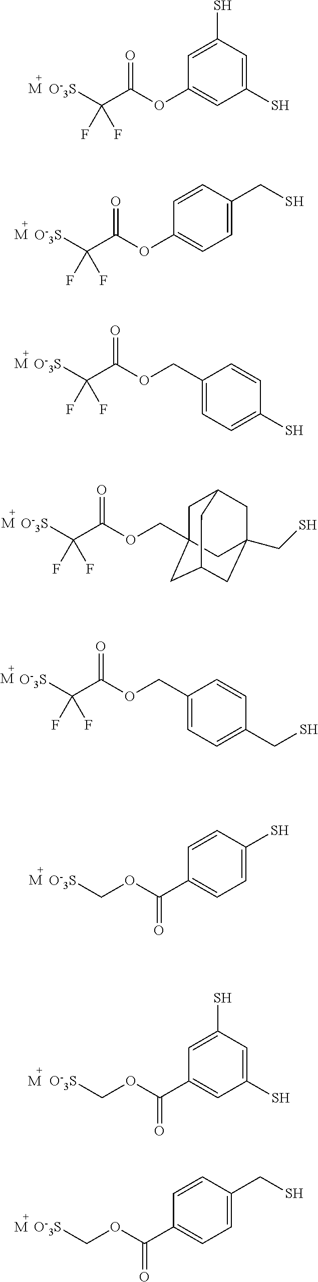

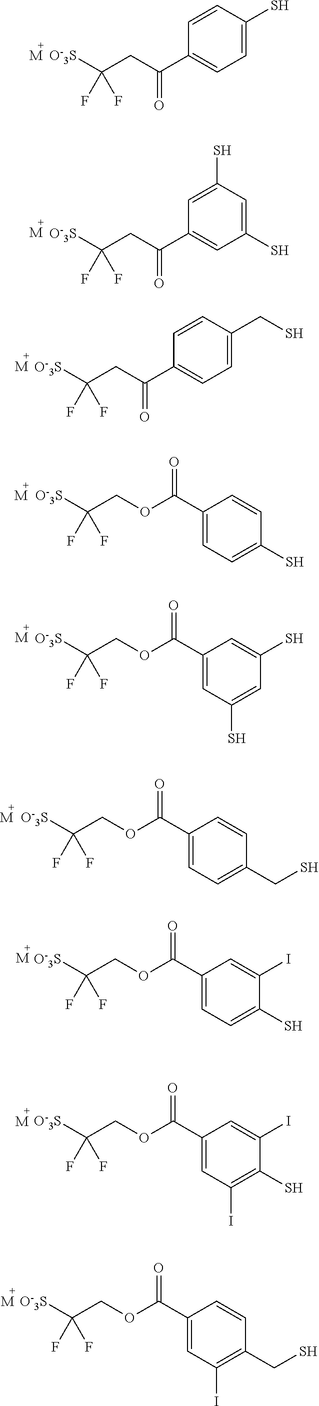

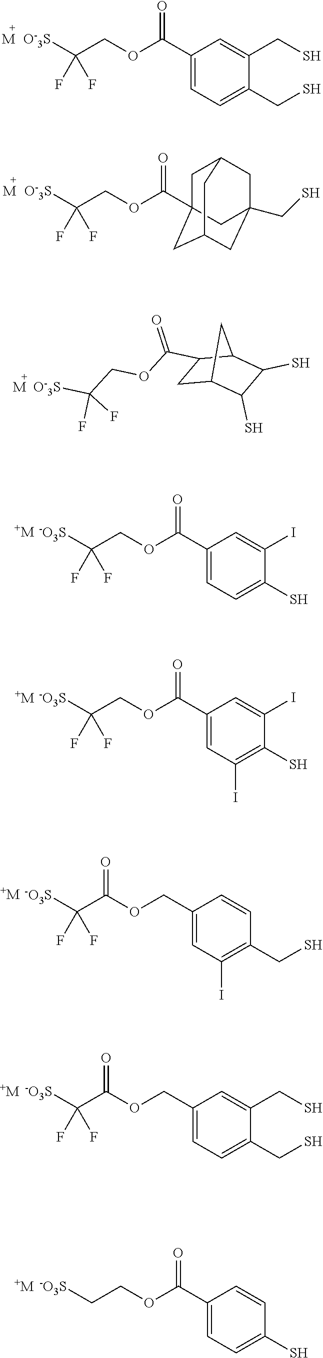

wherein in Group II, M⁺ is a counter cation.

Typically, extreme ultraviolet (EUV) light (13.5 nm) has a lower photon count compared to an ArF immersion light source. As a result, when the exposure dose is reduced, noise significantly increases in the boundary region between the exposed area and the unexposed area. In the case of a lithography process using an EUV light source, to compensate for this, a higher content of photoacid generators must be used compared to a lithography process using another light source with the same light intensity. However, if the resist composition contains a high content of photoacid generator, the glass transition temperature (Tg) of the base resin may change, and the thermal stability may deteriorate. Additionally, residual photoacid generators remaining during the EUV lithography process may degrade the resolution of the resist patterns.

The organic salt forms disulfide bonds due to secondary electrons during exposure, thereby generating additional acid and improving acid efficiency. Furthermore, because the anion of the organic salt has a relatively large molecular size, acid diffusion distance may be improved. As a result, the resist composition including the organic salt may exhibit enhanced developability and/or improved resolution.

### [Resist Composition]

A resist composition according to embodiments includes the above-described organic salt, a photoacid generator and a base resin.

In an embodiment, the organic salt may be included in an amount of about 0.1 parts to about 50 parts by weight based on 100 parts by weight of the base resin. In some embodiments, the organic salt may be included in an amount of about 0.1 parts to about 20 parts by weight based on 100 parts by weight of the base resin. When the above-described range is satisfied, an appropriate level of acid may be generated while reducing any performance loss, such as reduced uniformity and/or formation of foreign particles due to lack of solubility.

In an embodiment, the organic salt may be included in an amount of about 0.01 parts to about 70 parts by weight per 100 parts by weight of the photoacid generator. In some embodiments, the organic salt may be included in an amount of about 1 part to about 60 parts by weight per 100 parts by weight of the photoacid generator. When the above-described range is satisfied, the uniformity of the pattern may be improved while the formation of foreign particles due to insufficient solubility may be reduced.

The resist composition changes its solubility in the developer upon exposure to high-energy rays. The resist composition may be a positive resist composition in which an exposed portion of a resist film is dissolved and removed to form a positive resist pattern, or may be a negative resist composition in which an unexposed portion of a resist film is dissolved and removed to form a negative resist pattern. The resist composition may be a positive resist composition.

In addition, the resist composition according to an embodiment may use distilled water for the development process when forming a resist pattern, may be for an alkaline development process using an alkaline developer for the development process, or may be for a solvent development process using a developer containing an organic solvent (hereinafter, also referred to as an “organic developer”) for the development process.

The following describes optional components such as a base resin, photoacid generator, organic solvent, and quencher, which may be included as needed.

### <Base Resin>

The base resin may include a first repeating unit represented by Formula 3:

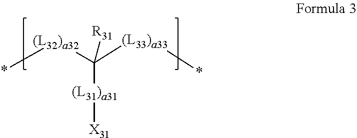

wherein in Formula 3,
L₃₁ to L₃₃ are each independently a single bond; O; S; C(═O); C(═O)O; OC(═O); C(═O)NR₃₂; NR₃₂C(═O); S(═O); S(═O)₂O; OS(═O)₂; or a linear, branched or cyclic C₁-C₃₀ divalent hydrocarbon group optionally including a heteroatom,
a31 to a33 are each independently an integer from 1 to 4,
R₃₁ and R₃₂ are each independently hydrogen; deuterium; a halogen atom; a cyano group; a hydroxyl group; an amino group; a carboxylate group; a thiol group; an ester moiety; a sulfonate moiety; a carbonate moiety; a lactone moiety; a sultone moiety; a carboxylic anhydride moiety; or a linear, branched or cyclic C₁-C₃₀ monovalent hydrocarbon group optionally including a heteroatom,
X₃₁ is an acid labile group, and
* is a bonding site with a neighboring atom.

For example, in Formula 3, L₃₁ to L₃₃ can each independently be a single bond; O; S; C(═O); C(═O)O; OC(═O); C(═O)NH; NHC(═O); S(═O); S(═O)₂O; OS(═O)₂; a substituted or unsubstituted C₁-C₃₀ alkylene group; a substituted or unsubstituted C₃-C₃₀ cycloalkylene group; a substituted or unsubstituted C₃-C₃₀ heterocycloalkylene group; a substituted or unsubstituted C₂-C₃₀ alkenylene group; a substituted or unsubstituted C₃-C₃₀ cycloalkenylene group; a substituted or unsubstituted C₃-C₃₀ heterocycloalkenylene group; a substituted or unsubstituted C₆-C₃₀ arylene group; or a substituted or unsubstituted C₁-C₃₀ heteroarylene group.

In some embodiments, in Formula 3, L₃₁ to L₃₃ may each independently be selected from a single bond; O; C(═O); C(═O)O; OC(═O); C(═O)NH; NHC(═O); and a C₁-C₂₀ alkylene group, a C₃-C₂₀ cycloalkylene group, a C₃-C₂₀ heterocycloalkylene group, a C₂-C₂₀ alkenylene group, a C₃-C₂₀ cycloalkenylene group, a C₃-C₂₀ heterocycloalkenylene group, a C₆-C₂₀ arylene group, and a C₁-C₂₀ heteroarylene group, each unsubstituted or substituted with a deuterium, a halogen atom, a cyano group, a hydroxyl group, an amino group, a carboxylate group, a thiol group, an ester moiety, a sulfonate moiety, a carbonate moiety, a carbamate moiety, a lactone moiety, a sultone moiety, a carboxylic anhydride moiety, a C₁-C₂₀ alkyl group, a C₁-C₂₀ halogenated alkyl group, a C₁-C₂₀ alkoxy group, a C₃-C₂₀ cycloalkyl group, a C₃-C₂₀ cycloalkoxy group, a C₆-C₂₀ aryl group, or a combination thereof.

In some embodiments, in Formula 3, L₃₁ to L₃₃ may each independently be selected from a single bond; O; C(═O); C(═O)O; OC(═O); C(═O)NH; NHC(═O); and a C₁-C₂₀ alkylene group, a C₃-C₂₀ cycloalkylene group, a C₃-C₂₀ heterocycloalkylene group, a phenylene group and a naphthylene group, each unsubstituted or substituted with deuterium, a halogen atom, a C₁-C₂₀ alkyl group, a C₁-C₂₀ halogenated alkyl group, a C₁-C₂₀ alkoxy group, a phenyl group, a naphthyl group, or a combination thereof.

In Formula 3, a31 to a33 represent the number of repetitions, and L₃₁ to L₃₃ represent the number of repetitions, respectively.

For example, in Formula 3, a31 to a33 may each independently be an integer from 1 to 3.

In some embodiments, in Formula 3, a31 to a33 may each independently be 1.

For example, in Formula 3, R₃₁ is hydrogen; deuterium; a halogen atom; a cyano group; a hydroxyl group; an amino group; a carboxylate group; a thiol group; and a C₁-C₂₀ alkyl group, a C₃-C₂₀ cycloalkyl group, and a C₆-C₂₀ aryl group, each unsubstituted or substituted with deuterium, a halogen atom, a cyano group, a hydroxyl group, an amino group, a carboxylate group, a thiol group, an ester moiety, a sulfonate moiety, a carbonate moiety, a carbamate moiety, a lactone moiety, a sultone moiety, a carboxylic anhydride moiety, a C₁-C₂₀ alkyl group, a C₁-C₂₀ halogenated alkyl group, a C₁-C₂₀ alkoxy group, a C₃-C₂₀ cycloalkyl group, a C₃-C₂₀ cycloalkoxy group, a C₆-C₂₀ aryl group, or a combination thereof.

In some embodiments, in Formula 3, R₃₁ may be selected from hydrogen; deuterium; a halogen atom; a cyano group; and a C₁-C₂₀ alkyl group unsubstituted or substituted with deuterium, a halogen atom, a cyano group, or a combination thereof.

In some embodiments, in Formula 3, R₃₁ may be H, D, F, CH₃, CH₂F, CHF₂, CF₃, CH₂CH₃, CHFCH₃, CHFCH₂F, CHFCHF₂, CHFCF₃, CF₂CH₃, CF₂CH₂F, CF₂CHF₂, CF₂CF₃, Cl, CH₂C₁, CHCl₂, CCl₃, CHClCH₃, CHClCH₂Cl, CHClCHCl₂, CHClCCl₃, CCl₂CH₃, CCl₂CH₂Cl, CCl₂CHCl₂, or CCl₂CCl₃.

For example, in Formula 3, R₃₂ may be hydrogen, deuterium, a halogen atom, a cyano group, a hydroxyl group, an amino group, a carboxylate group, a thiol group, a C₁-C₂₀ alkyl group, a C₁-C₂₀ halogenated alkyl group, a C₃-C₂₀ cycloalkyl group, or a C₆-C₂₀ aryl group.

As used herein, an acid-labile group refers to a group that is cleaved from the polymer by acid to generate a polar group, thereby facilitating the dissolution of the polymer in a developer, such as an aqueous TMAH solution.

For example, the acid dissociation constant (pKa) of the acid labile group may be about 13 or less, or about 3 to about 13, or about 5 to about 10 (calculated value).

For example, in Formula 3, X₃₁ may include a group having a tertiary bicyclic alkyl carbon, a group containing a tertiary alicyclic carbon, or an acetal.

In some embodiments, in Formula 3, X₃₁ may be represented by any one of Formulae 6-1 to 6-12:

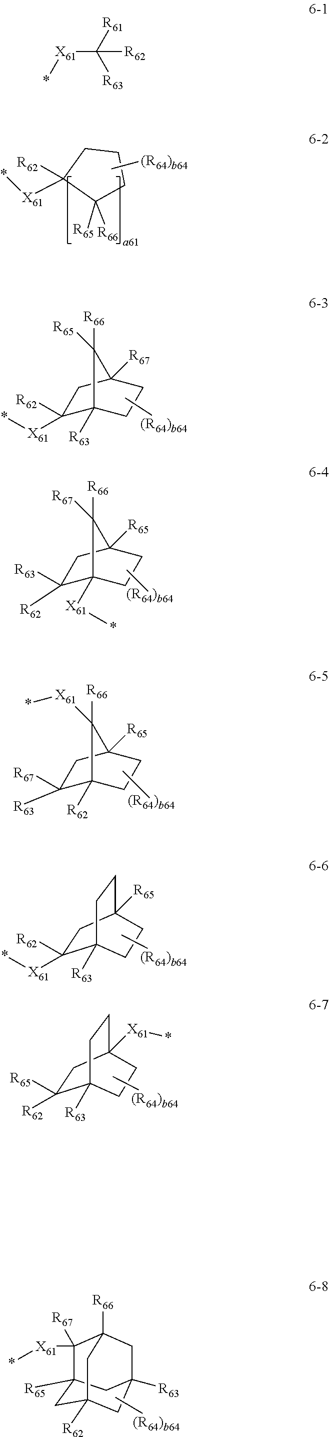

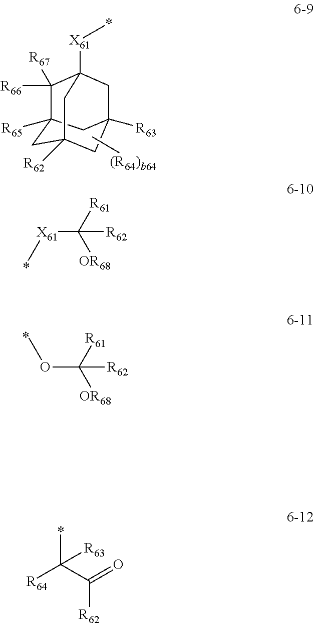

wherein in Formulae 6-1 to 6-12,
X₆₁ is an ester moiety, a sulfonate moiety, a carbonate moiety or a carbamate moiety;
a61 is selected from integers of 0 to 6;
R₆₁ and R₆₈ are each independently a linear, branched or cyclic C₁-C₂₀ monovalent hydrocarbon group optionally including in a heteroatom,
R₆₂ to R₆₇ are each independently hydrogen; deuterium; a halogen atom; a cyano group; a hydroxyl group; an amino group; a carboxylate group; a thiol group; an ester moiety; a sulfonate moiety; a carbonate moiety; a carbamate moiety; a lactone moiety; a sultone moiety; a carboxylic anhydride moiety; or a linear, branched or cyclic C₁-C₃₀ monovalent hydrocarbon group optionally including a heteroatom,
any two adjacent groups of R₆₁ to R₆₈ may optionally be bonded with each other to form a ring,
b64 is selected from integers of 1 to 10, and
* is a bonding site with a neighboring atom.

For example, in Formulae 6-1 to 6-12, X₆₁ may be an ester moiety or a carbonate moiety.

In some embodiments, in Formula 3, X₃₁ may be represented by any one of Formulae 6-21 to 6-46:

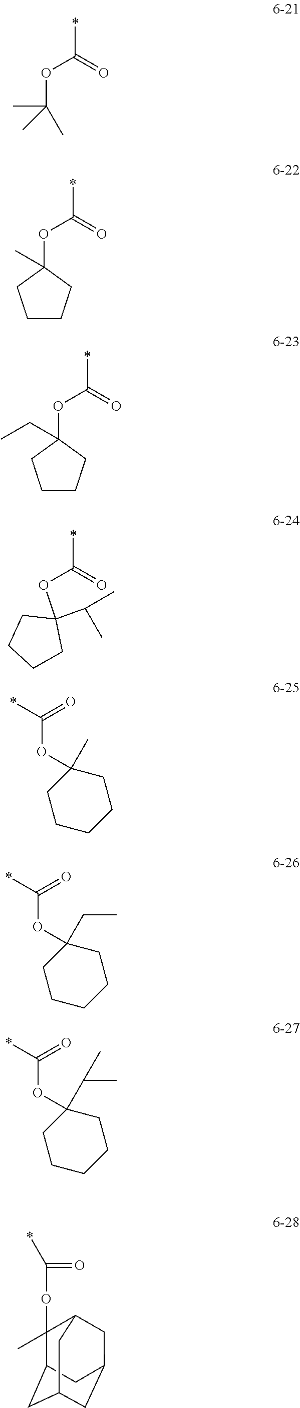

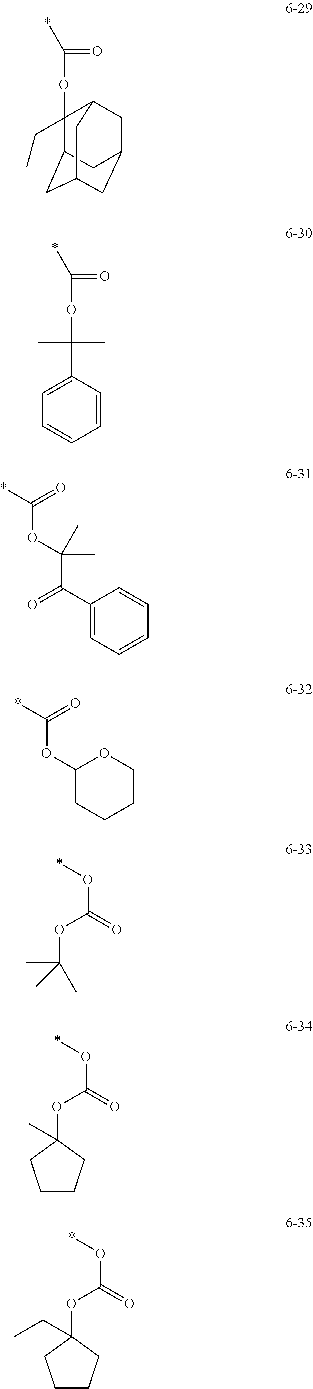

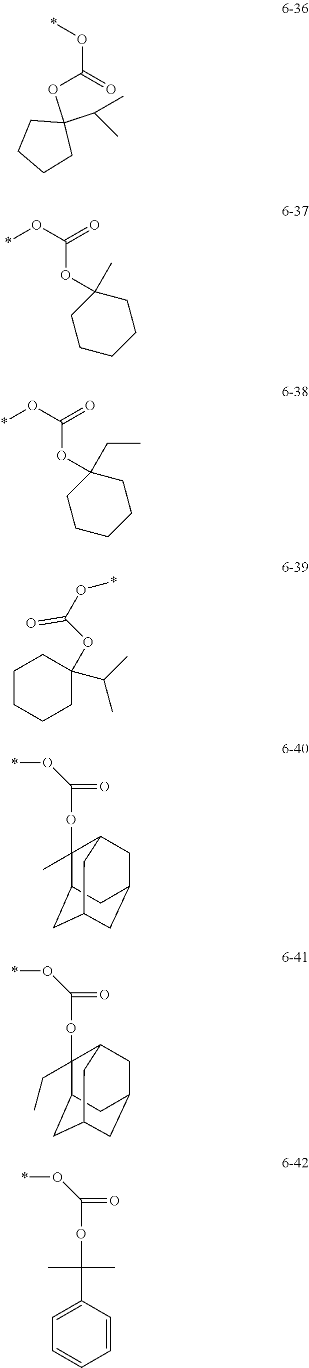

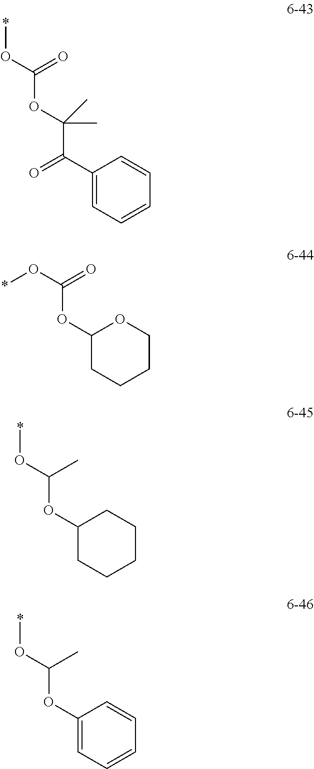 [SMILES: *OC(=O)OC(C)(C)C(=O)c1ccccc1 ; *OC(=O)OC1CCCCO1 ; *OC(C)OC1CCCCC1 ; *OC(C)Oc1ccccc1]

wherein in Formulae 6-21 to 6-46 above,
* is a bonding site with a neighboring atom.

In an embodiment, the first repeating unit may be selected from Group III:

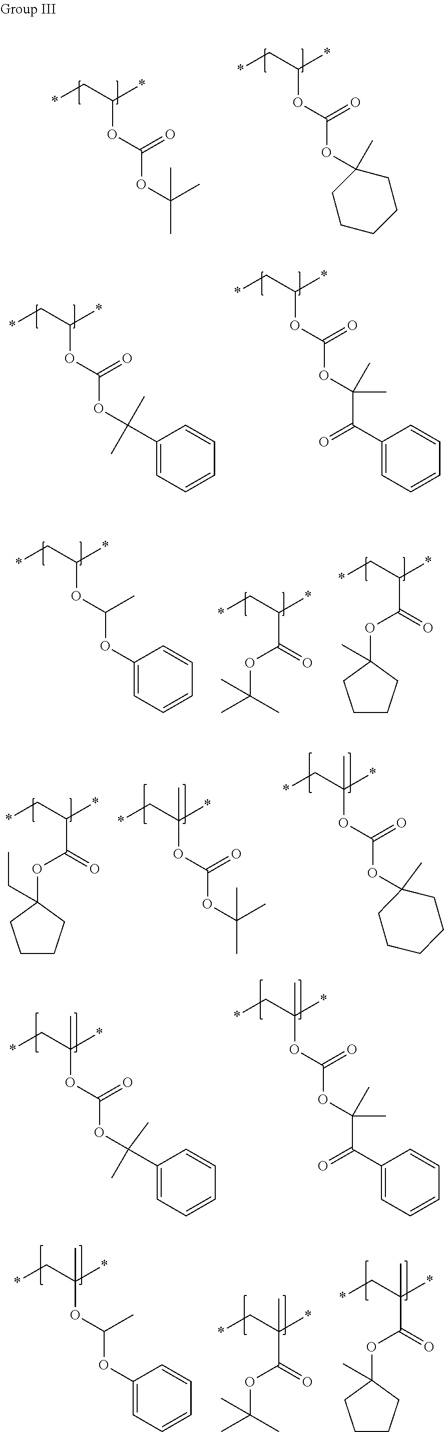

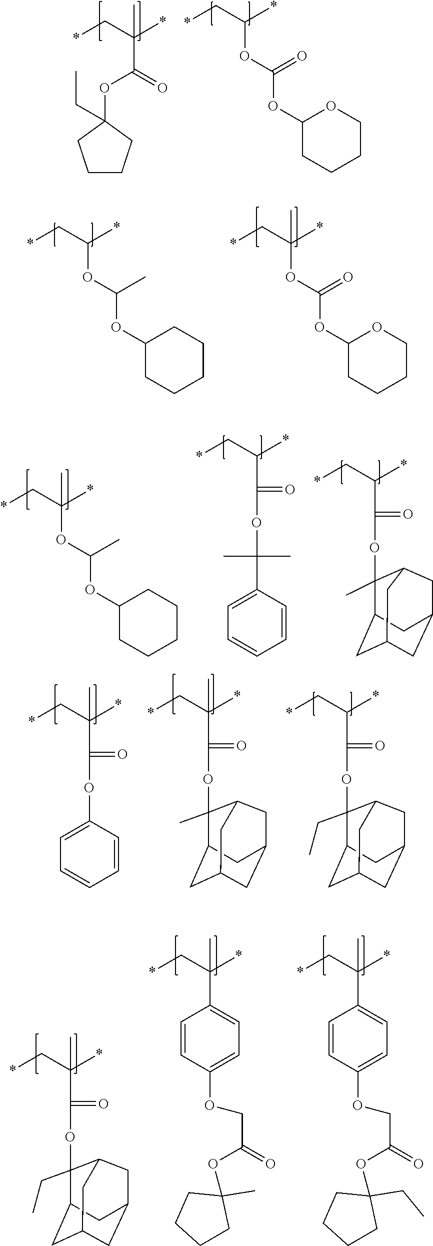

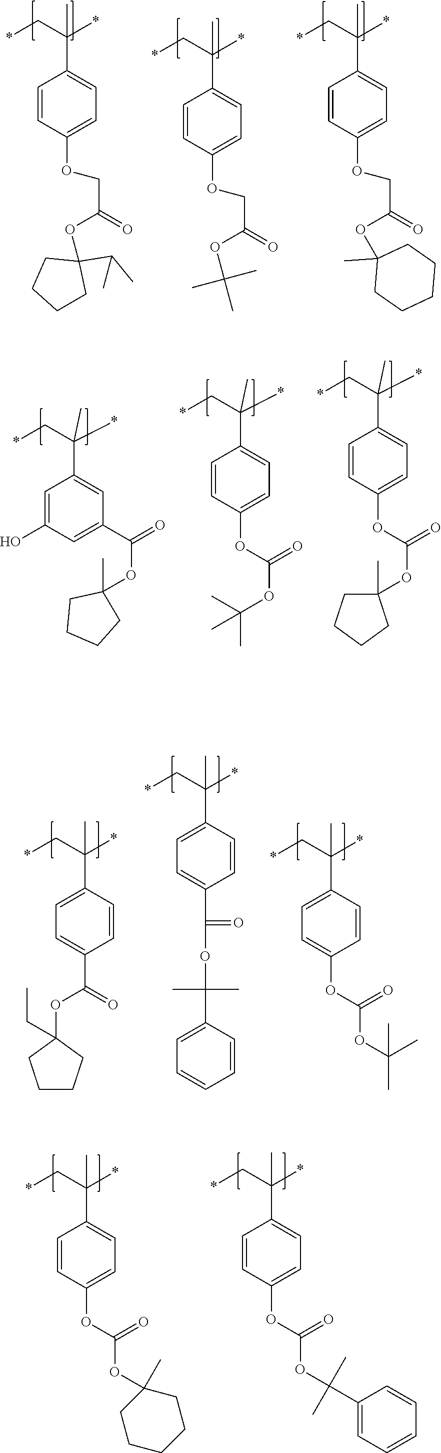

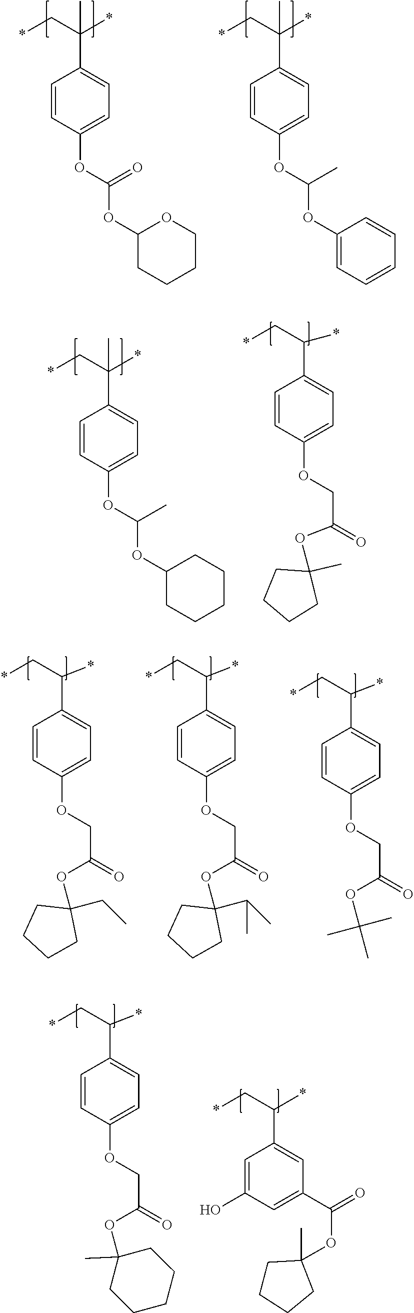

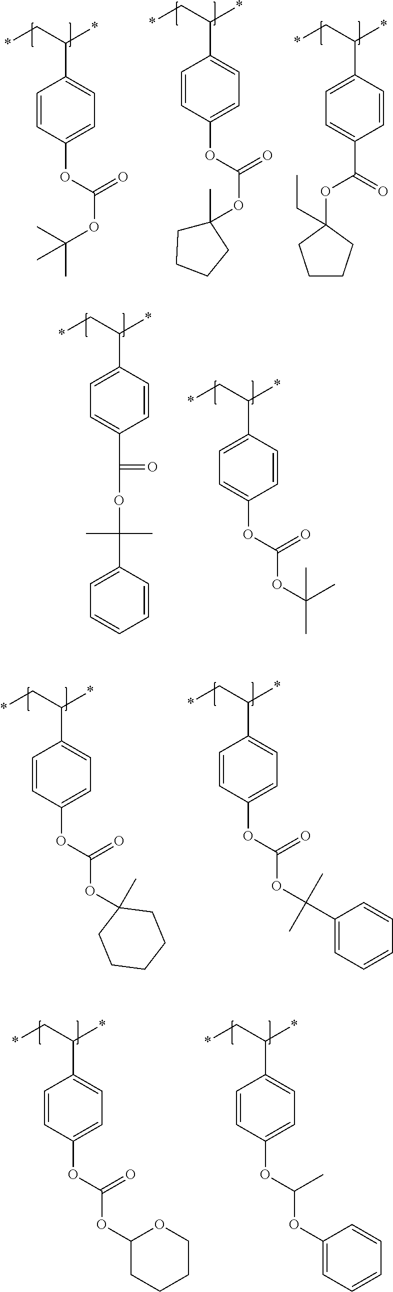

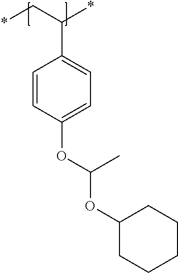

In an embodiment, the base resin may further include a second repeating unit represented by Formula 4:

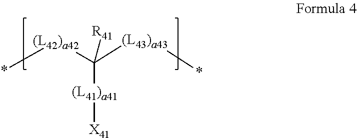

wherein in Formula 4,
L₄₁ to L₄₃ are each independently a single bond; O; S; C(═O); C(═O)O; OC(═O); C(═O)NR₄₂; NR₄₂C(═O); S(═O); S(═O)₂O; OS(═O)₂; or a linear, branched or cyclic C₁-C₃₀ divalent hydrocarbon group optionally including a heteroatom,
a41 to a43 are each independently an integer from 1 to 4,
R₄₁ and R₄₂ are each independently hydrogen; deuterium; a halogen atom; a cyano group; a hydroxyl group; an amino group; a carboxylate group; a thiol group; an ester moiety; a sulfonate moiety; a carbonate moiety; a lactone moiety; a sultone moiety; a carboxylic anhydride moiety; or a linear, branched or cyclic C₁-C₃₀ monovalent hydrocarbon group optionally including a heteroatom,
X₄₁ is a non-acid labile group, and
* is a bonding site with a neighboring atom.

For description of L₄₁ to L₄₃ in Formula 4, reference should be made to the description of L₃₁ in Formula 3.

For description of a41 to a43 in Formula 4, reference should be made to the description of a31 in Formula 3.

For description of R₄₁ in Formula 4, reference should be made to the description of R₃₁ in the Formula 3.

For description of R₄₂ in Formula 4, reference should be made to the description of R₃₂ in Formula 3.

For example, in Formula 4, X₄₁ may be hydrogen; deuterium; a halogen atom; a cyano group; a hydroxyl group; an amino group; a carboxylate group; a thiol group; or a linear, branched or cyclic C₁-C₃₀ monovalent hydrocarbon group optionally including one or more polar moieties selected from a halogen, a cyano group, a hydroxyl group, a carboxylate group, a thiol group, O, C═O, C(═O)O, OC(═O), S(═O)O, OS(═O), a lactone moiety, a sultone moiety and a carboxylic anhydride moiety.

In some embodiments, in Formula 4, X₄₁ may be selected from hydrogen, a hydroxyl group, and groups represented by Formulae 5-1 to 5-16:

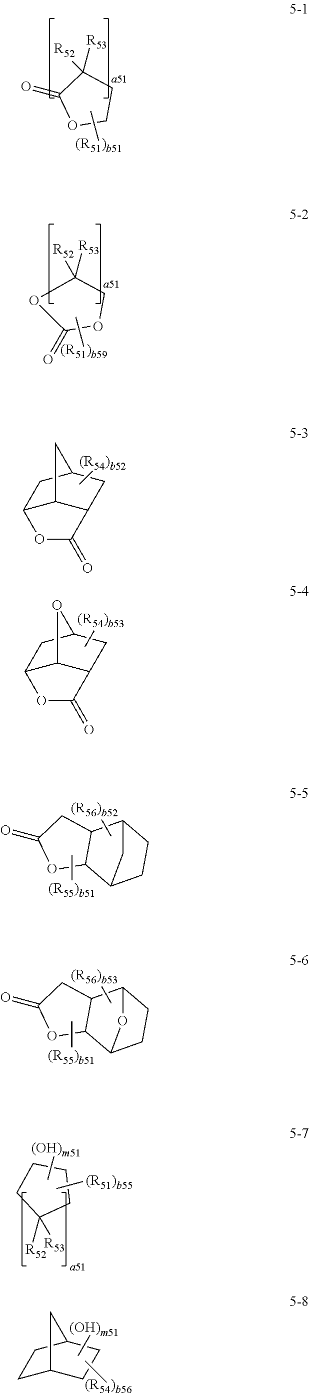

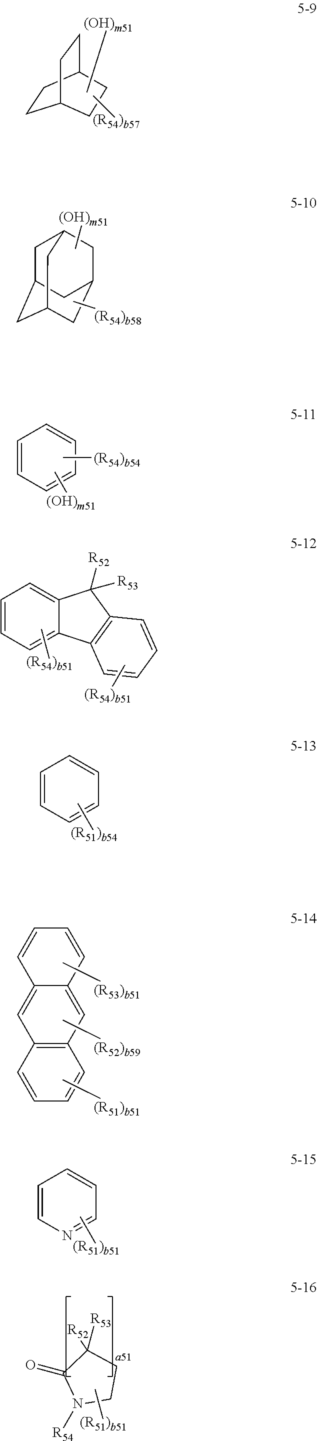

wherein in Formulae 5-1 to 5-16,
a51 is 1 or 2;
R₅₁ to R₅₆ are each independently a bonding site with an adjacent atom; hydrogen; deuterium; a halogen atom; a cyano group; a hydroxyl group; an amino group; a carboxylate group; a thiol group; a carbonyl moiety; an ester moiety; a sulfonate moiety; a carbonate moiety; a carbamate moiety; a lactone moiety; a sultone moiety; a carboxylic anhydride moiety; or a linear, branched or cyclic C₁-C₃₀ monovalent hydrocarbon group optionally including a heteroatom,
one of R₅₁ to R₅₃, one of R₅₄, and one of R₅₅ and R₅₆ is a bonding site with a neighboring atom,
b51 is selected from integers of 1 to 4,
b52 is selected from integers of 1 to 10,
b53 is selected from integers of 1 to 8,
b55 is selected from integers of 1 to 7,
b54 is selected from integers of 1 to 5,
b56 is selected from integers of 1 to 11,
b57 is selected from integers of 1 to 13,
b58 is selected from integers of 1 to 15,
b59 is selected from integers of 1 to 2, and
m51 is selected from integers of 1 to 4.

In some embodiments, in Formula 4, X₄₁ may be selected from a hydroxyl group and Formula 5-11.

In an embodiment, the second repeating unit may be selected from Group IV:

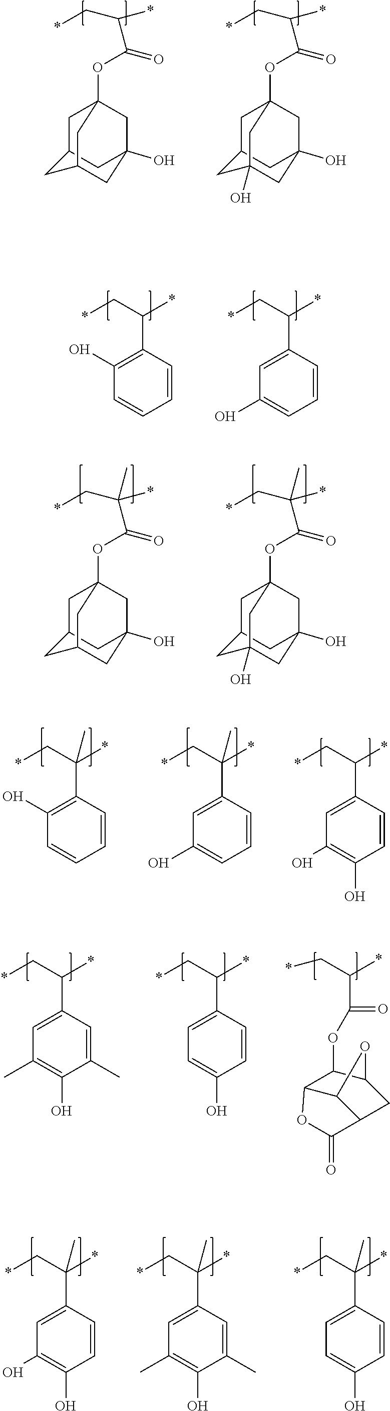

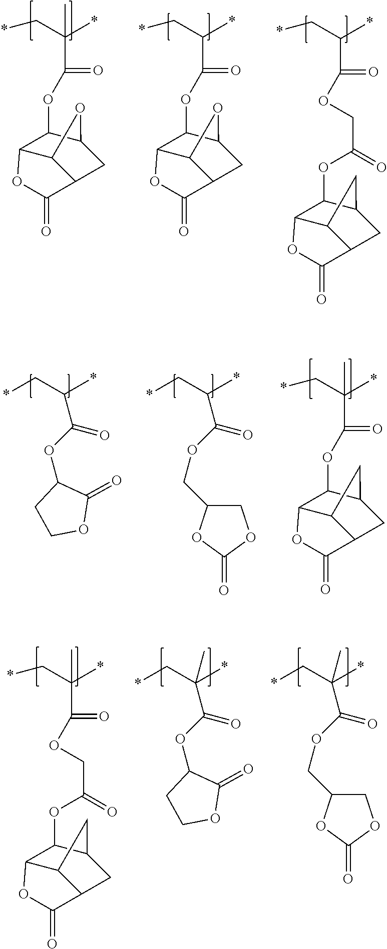

In an embodiment, the base resin may include about 1 mol % to about 100 mol % of the first repeating unit, or about 5 mol % to about 100 mol %, or about 10 mol % to about 100 mol %.

For example, the base resin may consist of the first repeating unit.

In another embodiment, the base resin may include about 0 mol % to about 99 mol % of the second repeating unit, or about 1 mol % to about 99 mol %, or about 10 mol % to about 90 mol %.

In another embodiment, the base resin may consist of the first repeating unit and the second repeating unit. For example, the base resin may contain about 1 mol % to about 99 mol % or about 10 mol % to about 90 mol % of the first repeating unit, and the base resin may contain about 1 mol % to about 99 mol % or about 10 mol % to about 90 mol % of the second repeating unit.

The base resin may have a weight average molecular weight (Mw) of about 1,000 to about 500,000, or about 3,000 to about 100,000, or about 5,000 to about 50,000, as measured by gel permeation chromatography using tetrahydrofuran solvent and polystyrene as standard materials.

The polydispersity index (PDI: Mw/Mn) of the base resin may be about 1.0 to about 3.0, or about 1.0 to about 2.5. Satisfying the above-described ranges may reduce the possibility of foreign matter remaining on the pattern, or minimize deterioration of the pattern profile. Accordingly, the resist composition may be more suitable for forming a fine pattern.

The base resin may be used as a single type or as a mixture of two or more different types.

### <Photoacid Generator>

The photoacid generator may be any compound capable of generating an acid when exposed to high-energy rays, such as UV, DUV, EB, EUV, X-rays, α-rays, or γ-rays.

The photoacid generator may include a sulfonium salt, an iodonium salt, and a combination thereof.

In an embodiment, the photoacid generator may be represented by Formula 7:

B₇₁_{⁺}A₇₁_{^{−}} Formula 7
wherein in Formula 7,
B₇₁_{⁺} is represented by Formula 7A, and A₇₁_{^{−}} is represented by any one of Formulae 7B to 7D,
B₇₁_{⁺} and A₇₁_{^{−}} may optionally be linked via a carbon-carbon covalent bond;

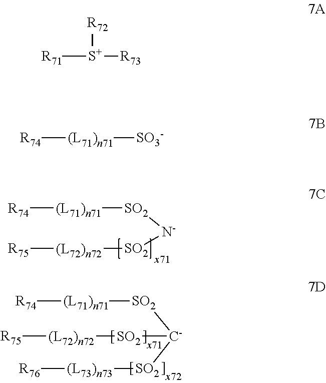

wherein in Formulae 7A to 7D,
L₇₁ to L₇₃ are each independently a single bond or CRR′,
R and R′ are each independently hydrogen, deuterium, a halogen atom, a cyano group, a hydroxyl group, a C₁-C₃₀ alkyl group, a C₁-C₃₀ halogenated alkyl group, a C₁-C₃₀ alkoxy group, a C₃-C₃₀ cycloalkyl group or a C₃-C₃₀ cycloalkoxy group,
n71 to n73 are each independently 1, 2 or 3,
x71 and x72 are each independently 0 or 1,
R₇₁ to R₇₃ are each independently a linear, branched or cyclic C₁-C₃₀ monovalent hydrocarbon group optionally including a heteroatom,
any two adjacent groups of R₇₁ to R₇₃ may optionally be bonded with each other to form a condensed ring,
R₇₄ to R₇₆ are each independently hydrogen; a halogen atom; or a linear, branched or cyclic C₁-C₃₀ monovalent hydrocarbon group optionally including a heteroatom.

For example, in Formula 7, B₇₁_{⁺} may be represented by Formula 7A, and A₇₁_{^{−}} may be represented by Formula 7B. In some embodiments, in Formula 7A, R₇₁ to R₇₃ may each be a phenyl group.

The photoacid generator may be included in an amount of about 0.01 parts to 40 parts by weight, about 0.1 parts to about 40 parts by weight, or about 0.1 parts to about 20 parts by weight based on 100 parts by weight of the polymer. When the above-described range is satisfied, appropriate resolution may be achieved, and issues related to foreign particle contamination during development or stripping may be reduced.

The photoacid generator may be used as a single type or as a mixture of two or more different types.

### <Quencher>

The resist composition may further include a quencher.

The quencher may be a salt that generates a weaker acid than the acid generated by the photoacid generator.

The quencher may include ammonium salts, sulfonium salts, iodonium salts, and combinations thereof.

In an embodiment, the quencher may be represented by Formula 8:

B₈₁_{⁺}A₈₁_{^{−}} Formula 8
wherein in Formula 8,
B₈₁_{⁺} is represented by any one of Formulae 8A to 8C, and A₈₁⁻ is represented by any one of Formulas 8D to 8F,
B₈₁_{⁺} and A₈₁_{^{−}} may optionally be linked via a carbon-carbon covalent bond;

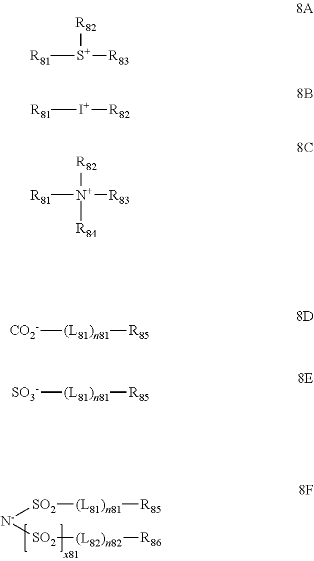

wherein in Formulae 8A to 8F,
L₈₁ and L₈₂ are each independently a single bond or CRR′,
R and R′ are each independently hydrogen, deuterium, a halogen atom, a cyano group, a hydroxyl group, a C₁-C₃₀ alkyl group, a C₁-C₃₀ halogenated alkyl group, a C₁-C₃₀ alkoxy group, a C₃-C₃₀ cycloalkyl group or a C₃-C₃₀ cycloalkoxy group,
n81 and n82 are each independently 1, 2 or 3,
x81 is 0 or 1,
R₈₁ to R₈₄ are each independently a linear, branched or cyclic C₁-C₃₀ monovalent hydrocarbon group optionally including a heteroatom,
any two adjacent groups of R₈₁ to R₈₄ may optionally be bonded with each other to form a condensed ring,
R₈₅ and R₈₆ are each hydrogen; a halogen atom; or a linear, branched or cyclic C₁-C₃₀ monovalent hydrocarbon group optionally including a heteroatom.

The quencher may be included in an amount of about 0 part to about 10 parts by weight, about 0.05 parts to about 5 parts by weight, or about 0.1 pars to about 3 parts by weight per 100 parts by weight of the polymer. When the above-described range is satisfied, appropriate resolution may be achieved, and issues related to foreign particle contamination during development or stripping may be reduced.

The quencher may be used as a single type or as a mixture of two or more different types.

### <Organic Solvent>

The organic solvent included in the resist composition is not particularly limited as long as it can dissolve or disperse optional components such as an organic salt, a base resin, a photoacid generator, and a quencher included as needed. The organic solvent may be used as a single type or as a mixture of two or more different types. Additionally, a mixed solvent including both water and an organic solvent may also be used.

Examples of organic solvents include, for example, alcohol-based solvents, ether-based solvents, ketone-based solvents, amide-based solvents, ester-based solvents, sulfoxide-based solvents, and hydrocarbon-based solvents.

In some embodiments, examples of the alcohol-based solvents may include: a monoalcohol-based solvent such as methanol, ethanol, n-propanol, isopropanol, 1-methoxy-2-propanol, 1-ethoxy-2-propanol, n-butanol, isobutanol, sec-butanol, tert-butanol, n-pentanol, isopentanol, 2-methylbutanol, sec-pentanol, tert-pentanol, 3-methoxybutanol, 3-methyl-3-methoxy butanol, n-hexanol, 2-methylpentanol, sec-hexanol, 2-ethylbutanol, 4-methyl-2-pentanol (MIBC), sec-heptanol, 3-heptanol, n-octanol, 2-ethylhexanol, sec-octanol, n-nonylalcohol, 2,6-dimethyl-4-heptanol, n-decanol, sec-undecyl alcohol, trimethylnonylalcohol, sec-tetradecyl alcohol, sec-heptadecyl alcohol, furfuryl alcohol, phenol, cyclohexanol, methylcyclohexanol, 3,3,5-trimethylcyclohexanol, benzyl alcohol, and diacetone alcohol; a polyalcohol-based solvent such as ethyleneglycol, 1,2-propylene glycol, 1,3-butylene glycol, 2,4-pentanediol, 2-methyl-2,4-pentanediol, 2,5-hexanediol, 2,4-heptanediol, 2-ethyl-1,3-hexanediol, diethyleneglycol, dipropyleneglycol, triethylene glycol, and tripropylene glycol; and a polyalcohol-containing ether-based solvent such as ethyleneglycol monomethylether, ethyleneglycol monoethylether, ethyleneglycol monopropylether, ethyleneglycol monobutylether, ethyleneglycol monohexylether, ethyleneglycol monophenylether, ethyleneglycol mono-2-ethylbutylether, diethyleneglycol monomethylether, diethyleneglycol monoethylether, diethyleneglycol monopropylether, diethyleneglycol monobutylether, diethyleneglycol monohexyl ether, diethylene glycol dimethylether, propylene glycol monomethylether, propylene glycol dimethylether, propylene glycol monoethylether, propylene glycol monopropylether, propylene glycol monobutylether, dipropyleneglycol monomethylether, dipropyleneglycol monoethylether, and dipropyleneglycol monopropylether.

Examples of the ether-based solvents may include: a dialkylether-based solvent such as diethylether, dipropylether, and dibutylether; a cyclic ether-based solvent such as tetrahydrofuran and tetrahydropyran; and an aromatic ring-containing ether-based solvent such as diphenylether and anisole.

Examples of the ketone-based solvents may include: a chain-shaped ketone-based solvent such as acetone, methylethylketone, methyl-n-propylketone, methyl-n-butylketone, methyl-n-pentylketone, diethylketone, methylisobutylketone, 2-heptanone, ethyl-n-butylketone, methyl-n-hexylketone, diisobutylketone, trimethylnonanone, and 2,6-dimethylheptan-4-one; a cyclic ketone-based solvent such as cyclopentanone, cyclohexanone, cycloheptanone, cyclooctanone, and methylcyclohexanone; and 2,4-pentanedione, acetonylacetone, and acetphenone.

Examples of the amide-based solvents may include: a cyclic amide-based solvent such as N,N′-dimethylimidazolidinone and N-methyl-2-pyrrolidone; and a chain-shaped amide-based solvent such as N-methylformamide, N,N-dimethylformamide, N,N-diethylformamide, acetamide, N-methylacetamide, N,N-dimethylacetamide, and N-methylpropyoneamide.

Examples of the ester-based solvents may include: an acetate ester-based solvent such as methyl acetate, ethyl acetate, n-propyl acetate, isopropyl acetate, n-butyl acetate, isobutyl acetate, sec-butyl acetate, t-butyl acetate, n-pentyl acetate, isopentyl acetate, sec-pentyl acetate, n-hexyl acetate, n-heptyl acetate, 3-methoxybutyl acetate, methylpentyl acetate, 2-ethylbutyl acetate, 2-ethylhexyl acetate, benzyl acetate, cyclohexyl acetate, methylcyclohexyl acetate, and n-nonyl acetate; a polyalcohol-containing ethercarboxylate-based solvent such as ethyleneglycol monomethylether acetate, ethyleneglycol monoethylether acetate, diethyleneglycol monomethylether acetate, diethyleneglycol monoethylether acetate, diethyleneglycol mono-n-butyl ether acetate, propylene glycol monomethylether acetate (PGMEA), propylene glycol monoethylether acetate, propylene glycol monopropylether acetate, propylene glycol monobutylether acetate, dipropylene glycol monomethylether acetate, and dipropylene glycol monoethylether acetate; a lactone-based solvent such as γ-butyrolactone and δ-valerolactone; a carbonate-based solvent such as dimethyl carbonate, diethyl carbonate, ethylene carbonate, and propylene carbonate; a lactate ester-based solvent such as methyl lactate, ethyl lactate, n-butyl lactate, and n-amyl lactate; and glycoldiacetate, methoxytriglycol acetate, ethyl propionate, n-butyl propionate, isoamyl propionate, diethyloxalate, di-n-butyloxalate, methyl acetoacetate, ethyl acetoacetate, diethyl malonate, dimethyl phthalate, and diethyl phthalate.

Examples of the sulfoxide-base solvents may include dimethyl sulfoxide and diethyl sulfoxide.

Examples of the hydrocarbon-based solvent may include aliphatic hydrocarbon solvents such as n-pentane, isopentane, n-hexane, isohexane, n-heptane, isoheptane, 2,2,4-trimethyl pentane, n-octane, isooctane, cyclohexane, and methylcyclohexane; and aromatic hydrocarbon solvents such as benzene, toluene, xylene, mesitylene, ethylbenzene, trimethylbenzene, methylethylbenzene, n-propylbenzene, isopropylbenzene, diethylbenzene, isobutylbenzene, triethylbenzene, diisopropylbenzene, and n-amylnaphthalene.

In some embodiments, the organic solvent may be selected from alcohol-based solvents, amide-based solvents, ester-based solvents, sulfoxide-based solvents, and a combination thereof. In some embodiments, the solvent may be selected from propylene glycol monomethyl ether, propylene glycol monoethyl ether, propylene glycol monomethyl ether acetate, N-methyl-2-pyrrolidone, N,N-dimethylacetamide, ethyl lactate, dimethyl sulfoxide and a combination thereof.

Meanwhile, if an acid-labile group in acetal form is used, a high-boiling alcohol, such as diethylene glycol, propylene glycol, glycerol, 1,4-butanediol, or 1,3-butanediol, may be additionally added to accelerate the deprotection reaction of the acetal.

The solvent may be used in an amount of about 200 parts to about 20,000 parts by weight or about 2,000 parts to about 10,000 parts by weight, based on 100 parts by weight of the polymer.

### <Optional Component>

The resist composition may further a surfactant, a crosslinking agent, a leveling agent, a colorant, or a combination thereof, as needed.

The resist composition may further include a surfactant to improve coatability, developability, and the like. Examples of the surfactant may include a nonionic surfactant such as polyoxyethylene lauryl ether, polyoxyethylene stearyl ether, polyoxyethyleneoleyl ether, polyoxyethylene n-octylphenyl ether, polyoxyethylene n-nonylphenyl ether, polyethyleneglycol dilaurate, and polyethyleneglycol distearate. Any commercially available product or a synthetic product may be used as the surfactant. Surfactants can be either commercially available or synthetic. Examples of the commercially available product may include KP341 (manufactured by Shin-Etsu Chemical Co., Ltd.), Polyflow No. 75 and Polyflow No. 95 (manufactured by Kyoeisha Chemical Co., Ltd.), Eftop EF301, Eftop EF303, and Eftop EF352 (manufactured by Mitsubishi Materials Electronic Chemicals Co., Ltd.), MEGAFACE® F171, MEGAFACE F173, R40, R41, and R43 (manufactured by DIC Corporation), Fluorad® FC430, Fluorad FC431 (manufactured by 3M Co., Ltd.), AsahiGuard AG710 (manufactured by AGC Co., Ltd.), and Surflon® S-382, Surflon SC-101, Surflon SC-102, Surflon SC-103, Surflon SC-104, Surflon SC-105, and Surflon SC-106 (manufactured by AGC Seimi Chemical Co., Ltd).

The surfactant may be included in an amount of about 0 part to about 20 parts by weight based on 100 parts by weight of the polymer.

The surfactants may be used as a single type or as a mixture of two or more different types.

The method of producing the resist composition is not particularly limited, and for example, any method of mixing a polymer and optional components added as needed in a solvent may be used. Temperature or time in the mixing is not particularly limited. If necessary, filtration may be performed after the mixing.

### [Pattern Formation Method]

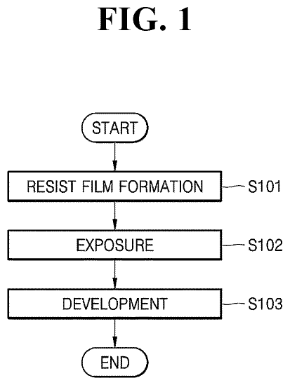

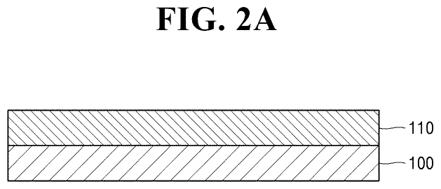

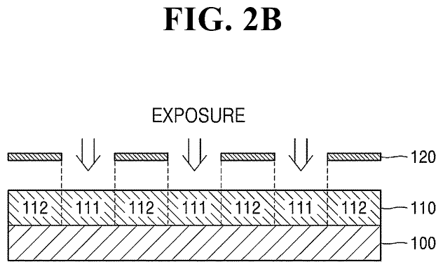

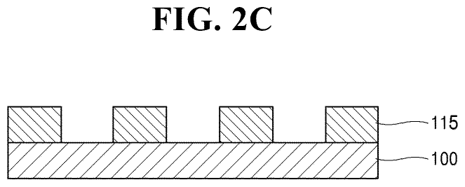

Hereinafter, a method of forming a pattern according to embodiments will be described in more detail with reference to FIG. 1 and FIGS. 2A to 2C. FIG. 1 is a flowchart illustrating a method of forming a pattern according to embodiments, and FIGS. 2A to 2C are cross-sectional side views illustrating a method of forming a pattern according to embodiments. Hereinafter, a method of forming a pattern by using a positive resist composition will be described by way of an example, but the embodiment is not limited thereto.

Referring to FIGS. 1 and 2A to 2C, a method of forming a pattern includes applying a resist composition to form a resist film (S101), exposing at least a portion of the resist film to high-energy rays (S102) to provide an exposed resist film, and developing the exposed resist film using a developer (S103). These operations may be omitted or may be performed in a different order, if necessary.

First, a substrate 100 is prepared. The substrate 100 may be a semiconductor substrate such as a silicon substrate and a germanium substrate, or may be formed of glass, quartz, ceramic, copper, or the like. In some embodiments, the substrate 100 may include Groups III to V compounds, such as GaP, GaAs, and GaSb.

A resist film 110 may be formed on the substrate 100 by applying the resist composition thereto to a desired thickness using a coating method. If necessary, a post application bake (PAB) may be performed to remove the organic solvent remaining in the resist film 110.

As the coating method, spin coating, dipping, roller coating, or other common coating methods may be used. Among them, spin coating may be used in particular, and the resist film 110 having a desired thickness may be formed by adjusting viscosity, concentration, and/or spin speed of the resist composition. In some embodiments, the resist film 110 may have a thickness of about 10 nm to about 300 nm. In some embodiments, the resist film 110 may have a thickness of about 30 nm to about 200 nm.

The lower limit of a PAB temperature may be 60° C. or higher, or 80° C. or higher. In addition, the upper limit of the PAB temperature may be 150° C. or less, or 140° C. or lower. The lower limit of a PAB time may be 5 seconds or more, or 10 seconds or more. The upper limit of the PAB time may be 600 seconds or less, or 300 seconds or less.

Before applying the resist composition on the substrate 100, a film to be etched (not shown) may be also formed on the substrate 100. The film to be etched may refer to a layer onto which an image is transferred from a resist pattern and converted into a pattern. In an embodiment, the film to be etched may be formed to include, for example, an insulating material such as a silicon oxide, a silicon nitride, and a silicon oxynitride. In some embodiments, the film to be etched may be formed to include a conductive material such as a metal, a metal nitride, a metal silicide, and a metal silicide nitride film. In some embodiments, the film to be etched may be formed to include a semiconductor material such as polysilicon.

In an embodiment, an anti-reflection film may further be formed on the substrate 100 to improve and/or maximize efficiency of the resist. The anti-reflection film may be an organic or inorganic anti-reflection film.

In an embodiment, a protective film may further be formed on the resist film 110 to reduce effects of alkaline impurities included during a process. In addition, in the case of performing immersion lithography, a protective film for immersion lithography may be formed on the resist film 110 to avoid direct contact between an immersion medium and the resist film 110.

Subsequently, at least a portion of the resist film 110 may be exposed to high-energy rays. For example, high-energy rays having passed through a mask 120 may reach at least one portion of the resist film 110. Therefore, the resist film 110 may have an exposed portion 111 and an unexposed portion 112.

Although not limited to a specific theory, it is believed that the additives may react with each other upon exposure to radiation, forming disulfide bonds, which may lead to the generation of additional acid. As a result, even when using the same or a reduced amount of photoacid generator, a higher-quality pattern may be achieved.

In some cases, the exposure may be performed by irradiating high-energy rays through a mask with a certain pattern by using a liquid such as water as a medium. Examples of the high-energy rays may include electromagnetic waves such as ultraviolet rays, deep ultraviolet (DUV) rays, extreme ultraviolet (EUV) rays (wavelength of 13.5 nm), X-rays, and γ-rays; and charged particle beams such as electron beams (EBs) and a particle beams. Irradiation of these high-energy rays may be collectively referred to as “exposure.”

Various light sources may be used for the exposure, for example, a light source emitting laser beams in the UV range, such as a KrF excimer laser (wavelength of 248 nm), an ArF excimer laser (wavelength of 193 nm), and an F₂ excimer laser (wavelength of 157 nm), a light source emitting harmonic laser beams in the far ultraviolet or vacuum ultraviolet range by converting wavelengths of laser beams received from a solid laser light source (YAG or semiconductor laser), and a light source emitting EBs or EUVs may be used. During exposure, the exposure may be usually performed through a mask corresponding to a desired pattern, but when exposure light is an EB, the exposure may be performed through direct writing without using a mask.

The integrated dose of high-energy rays, for example, when using extreme ultraviolet rays as high-energy rays, may be 2000 mJ/cm² or less, or 500 mJ/cm² or less. In addition, when EBs are used as the high-energy rays, the integral dose may be 5,000 μC/cm² or less, or 1,000 μC/cm² or less.

Additionally, a post exposure bake (PEB) may be performed. The lower limit of the temperature of PEB may be 50° C. or more, or 80° C. or more. The upper limit of the PEB temperature may be 180° C. or lower, or 130° C. or lower. The lower limit of the time of the PEB time may be 5 seconds or more, or 10 seconds or more. The upper limit of the time of the PEB may be 600 seconds or less, or 300 seconds or less.

Next, the exposed resist film 110 may be developed using a developer to form a resist pattern 115. At this time, the exposed portion (111) can be washed away and removed by the developer, and the unexposed portion (112) remains without being washed away by the developer.

Examples of the developer may include distilled water, an alkaline developer, and a developer including an organic solvent (hereinafter also referred to as “organic developer”). Examples of a development method may include a dipping method, a puddle method, a spray method, and a dynamic injection method. The developing temperature may be, for example, about 5° C. or more and about 60° C. or less, and a developing time may be, for example, about 5 seconds or more and about 300 seconds or less.

Examples of the alkaline developer may include an alkaline aqueous solution in which one or more alkaline compounds such as sodium hydroxide, potassium hydroxide, sodium carbonate, sodium silicate, sodium metasilicate, aqueous ammonia, ethylamine, n-propylamine, diethylamine, di-n-propylamine, triethylamine, methyldiethyamine, ethyldimethylamine, triethanolamine, tetramethyl ammonium hydroxide (TMAH), pyrrole, piperidine, choline, 1,8-diazabicyclo[5.4.0]-7-undecene (DBU), and 1,5-diazabicyclo[4.3.0]-5-nonene (DBN) are dissolved. The alkaline developer may further include a surfactant.

The lower limit of an amount of the alkaline compound included in the alkaline developer may be about 0.1 wt % or more, or about 0.5 wt % or more, or about 1 wt % or more. Additionally, the upper limit of the amount of the alkaline compound included in the alkaline developer may be about 20 wt % or less, or about 10 wt % or less, or about 5 wt % or less.

After development, the resist pattern may be washed with ultrapure water, and then any water remaining on the substrate and pattern may be removed.

Examples of the organic solvent included in the organic developer may include the same organic solvents as those exemplified in the part of <Organic solvent> of [Resist composition].

In some embodiments, nBA (n-butyl acetate), PGME, PGMEA, ethyl lactate, GBL (γ-butyrolactone), IPA (isopropanol), etc. may be used as the organic developer. The organic developer may further contain an organic acid such as acetic acid, formic acid, citric acid, etc.

The lower limit of the organic solvent content in the organic developer may be about 80 wt % or more, about 90 wt % or more, or about 95 wt % or more, and especially about 99 wt % or more.

In an embodiment, the developer may include distilled water, an alkaline developer, or a combination thereof, and the exposed portion 111 may be removed by the developer.

The organic developer may also include surfactants. Additionally, organic developer may contain trace amounts of moisture. Additionally, development may be stopped by substituting a different type of solvent from the organic developer during development.

The resist pattern after development may be further cleaned. Cleaning solutions such as ultrapure water and rinse solution may be used. There are no particular restrictions on the rinse solution as long as it does not dissolve the resist pattern, and common solutions containing organic solvents may be used. For example, the rinse solution may be an alcohol-based solvent or an ester-based solvent. After cleaning, any remaining rinse solution on the substrate and the resist pattern may be removed. When ultrapure water is used, any remaining water on the substrate and pattern may be removed.

Additionally, the developer may be used alone or in combination of two or more types.

As described above, after forming the resist pattern, a patterned wiring substrate may be obtained by etching. The etching method can be performed by a known method, such as dry etching using plasma gas, and wet etching using an alkaline solution, a copper (II) chloride solution, an iron (II) chloride solution, etc.

After forming the resist pattern, plating may also be performed. The plating method is not particularly limited, but examples include copper plating, solder plating, nickel plating, and gold plating.

The remaining resist pattern after etching may be stripped using an organic solvent. Examples of such organic solvents may include, but are not limited to, propylene glycol monomethyl ether acetate (PGMEA), propylene glycol monomethyl ether (PGME), and ethyl lactate (EL). The stripping method is not particularly limited and may include, for example, immersion methods and spray methods. Additionally, the wiring substrate with the resist pattern formed may be a multilayer wiring substrate and may have small-diameter through holes.

In an embodiment, the wiring substrate is formed by depositing metal in a vacuum after forming the resist pattern, and then dissolving the resist pattern in a solution, a method known as the lift-off method.

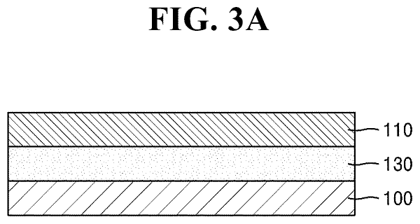

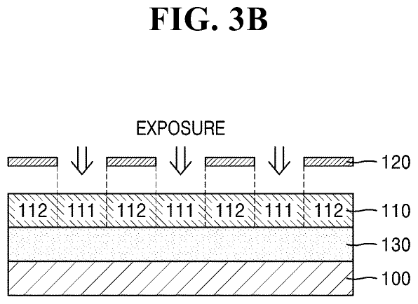

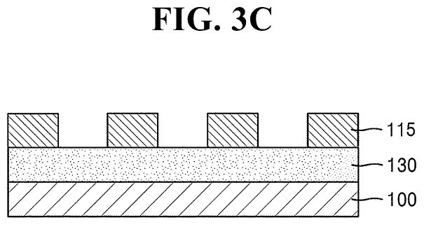

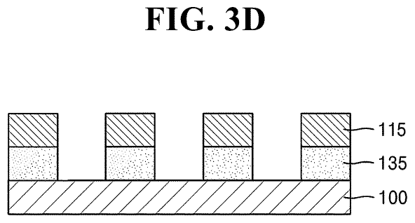

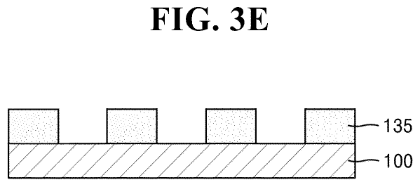

FIGS. 3A to 3E are cross-sectional side views showing a method of forming a patterning structure according to an embodiment.

As shown in FIG. 3A, a material layer 130 may be formed on the substrate 100 before forming the resist film 110 on the substrate 100. The resist film 110 may be formed on top of the material layer 130. The material layer 130 may include an insulating material (e.g., silicon oxide, silicon nitride), a semiconductor material (e.g., silicon), or a metal (e.g., copper). In some embodiments, the material layer 130 may have a multi-layer structure. The material of the material layer 130 may be different from the material of the substrate 100.

As shown in FIG. 3B, the resist film 110 may undergo a prebake process before exposure and then be exposed to high-energy rays through the mask 120, and subsequently the resist film 110 may include an exposed portion 111 and an unexposed portion 112.

As shown in FIG. 3C, the exposed resist film 110 may be developed using a developer. The exposed portion 111 may be washed away by the developer, while the unexposed portion 112 remain without being washed away by the developer.

As shown in FIG. 3D, the resist pattern 115 may serve as a mask for etching the exposed portions of the material layer 130 to form the material pattern 135 on the substrate 100.

As shown in FIG. 3E, the resist pattern 115 may be removed.

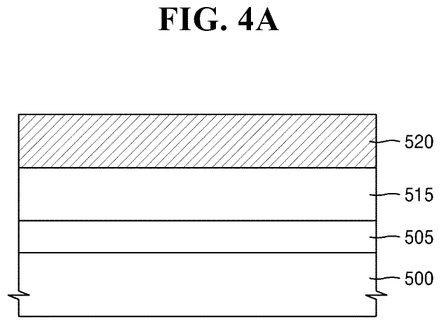

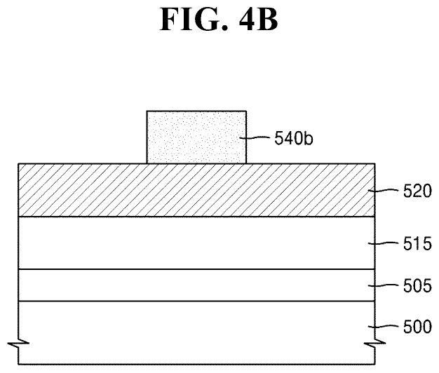

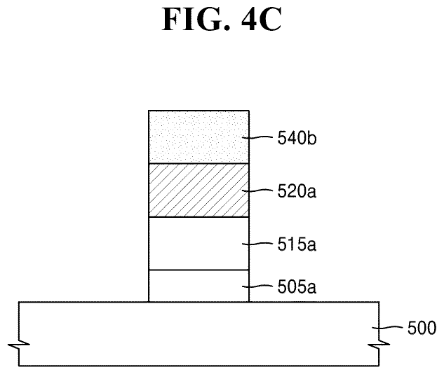

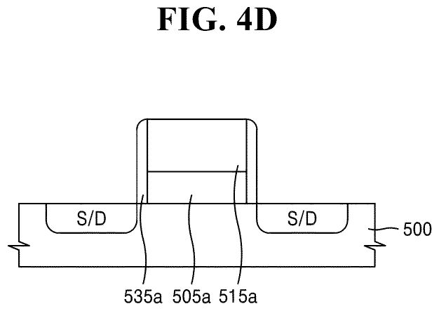

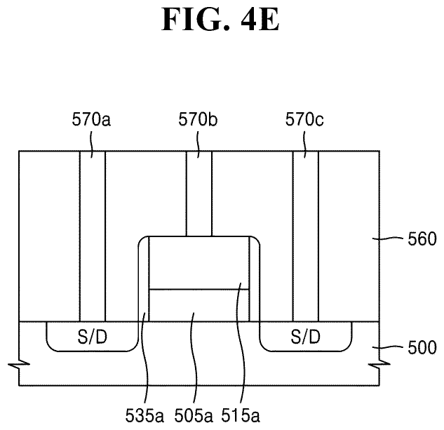

FIGS. 4A to 4E are cross-sectional side views showing a method of forming a semiconductor device according to an embodiment.

As shown in FIG. 4A, a gate dielectric 505 (e. g., silicon oxide) may be formed on the substrate 500. The substrate 500 may be a semiconductor substrate such as a silicon substrate. A gate layer 515 (e.g., doped polysilicon) may be formed on gate dielectric 505. A hardmask layer 520 may be formed on the gate layer 515.

As shown in FIG. 4B, a resist pattern 540b may be formed on the hardmask layer 520. The resist pattern 540b may be formed using a resist composition according to an embodiment. The resist composition may include an organic solvent.

As shown in FIG. 4C, the gate layer 515 and the gate dielectric 505 may be etched to form a hardmask pattern 520a, a gate electrode pattern 515a, and a gate dielectric pattern 505a.

As shown in FIG. 4D, a spacer layer may be formed on the gate electrode pattern 515a and the gate dielectric pattern 505a. The spacer layer may be formed using a deposition process (e.g., CVD). The spacer layer may be etched to form spacers 535a (e.g., silicon nitride) on the sidewalls of the gate electrode pattern 515a and the gate dielectric pattern 505a. After forming the spacers 535a, ions may be implanted into the substrate 500 to form source/drain impurity regions (S/D).

As shown in FIG. 4E, an interlayer insulating film 560 (e.g., an oxide) may be formed on the substrate 500, covering the gate electrode pattern 515a, gate dielectric pattern 505a, and spacers 535a. Subsequently, the interlayer insulating film 560 may have electrical contacts 570a, 570b, and 570c formed to connect with the gate electrode pattern 515a and the S/D regions. The electrical contacts 570a, 570b, and 570c may be formed of a conductive material (e.g., metal). Although not shown, a barrier layer may be formed between the sidewalls of the interlayer insulating film 560 and the electrical contacts 570a, 570b, and 570c.

FIGS. 4A to 4E show examples of forming transistors, but the disclosure is not limited thereto.

The resist composition according to an embodiment may be used in the patterning process to form other types of semiconductor devices.

For example, although not illustrated in FIGS. 4D and 4E, in some embodiments, the hard mask pattern 520a may not be removed before the spacer 535a is formed. For example, if the hard mask pattern 520a is not removed, then the hard mask pattern 520a may remain on top of the gate electrode pattern 515a in FIGS. 4D and 4E, the spacer 535a may cover a sidewall of the hard mask pattern 520a in FIGS. 4D and 4E, and the electrical contact 570b may extend through an opening in the hard mask pattern 520a to directly contact an upper surface of the gate electrode 515a.

While the disclosure will be described in more detail using the following examples and comparative examples, the technical scope of the disclosure is not limited to these examples.

### EXAMPLES

### Synthesis Example 1: Synthesis of Organic Salt 1

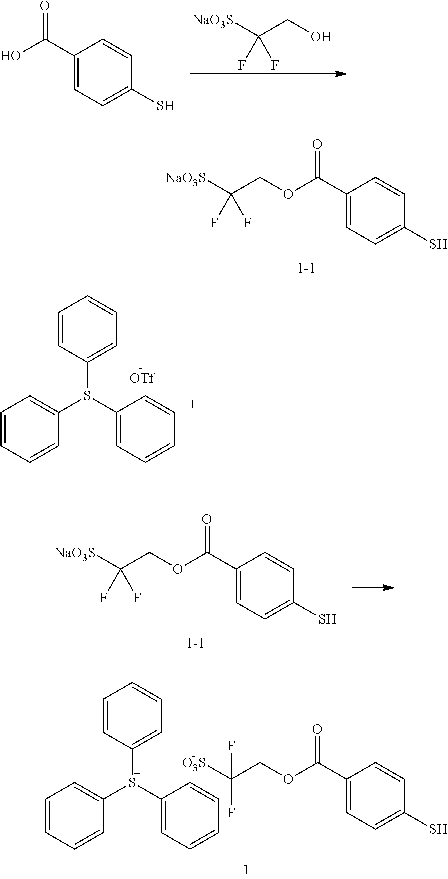

### (1) Synthesis of Intermediate 1-1

4-mercaptobenzoic acid (0.776 g, 5.03 mmol) and sodium 1,1-difluoro-2-hydroxyethanesulfonate (0.926 g, 5.03 mmol) were dissolved in 50 mL of dichloromethane and 1 mL of dimethylformamide. Then, dicyclohexylcarbodiimide (1.141 g, 5.53 mmol), 4-dimethylaminopyridine (0.673 g, 5.53 mmol), and triethylamine (0.78 mL, 5.53 mmol) were added, and the reaction mixture was stirred at room temperature for 16 hours. Upon completion of the reaction, the precipitate was removed by filtration, and the organic layer was washed twice with saturated brine. After collecting the organic layer and removing the solvent, the crude product was purified by column chromatography using a dichloromethane (DCM):methanol (MeOH)=20:1 (v:v) eluent system.

The purified compound was then dissolved in 30 mL of tetrahydrofuran (THF), and sodium borohydride (0.572 g, 15.09 mmol) was slowly added at 0° C. while stirring for 2 hours. The reaction was continued at room temperature for 4 hours. After the reaction was complete, the solvent was removed, and the residue was dissolved in DCM. The organic layer was washed three times with 0.1 M hydrochloric acid solution and three times with water, followed by drying over magnesium sulfate. The solvent was then removed from the organic layer, yielding Intermediate 1-1 (0.805 g, 49%). The obtained compound was confirmed using ¹H-NMR and LC-MS.

¹H-NMR (500 MHz, CD₂Cl₂): δ 7.89 (d, 2H), 7.50 (d, 2H), 4.63 (t, 2H), 3.34 (s, 1H),

LC-MS m/z=296.97 (anion).

### (2) Synthesis of Organic Salt 1

Intermediate 1-1 (0.32 g, 1 mmol) and triphenylsulfonium triflate (0.41 g, 1 mmol) were mixed in 10 mL of dichloromethane and 1 mL of water and stirred for 4 hours. Afterward, the organic layer was separated, dried over MgSO₄, and filtered. The filtrate was concentrated under reduced pressure, and the obtained residue was purified by silica gel column chromatography to afford Organic Salt 1 (0.38 g, 67.9%). The obtained compound was confirmed using ¹H-NMR and MALDI.

¹H-NMR (500 MHz, CD₂Cl₂): δ 7.89 (d, 2H), 7.75 (m, 15H), 7.50 (d, 2H), 4.63 (t, 2H), 3.34 (s, 1H),

HRMS (MALDI) calculated for C₂_{₇}H₂₂F₂O₅S₃: m/z 560.06, found: 560.05.05.

### Synthesis Example 2: Synthesis of Organic Salt 2

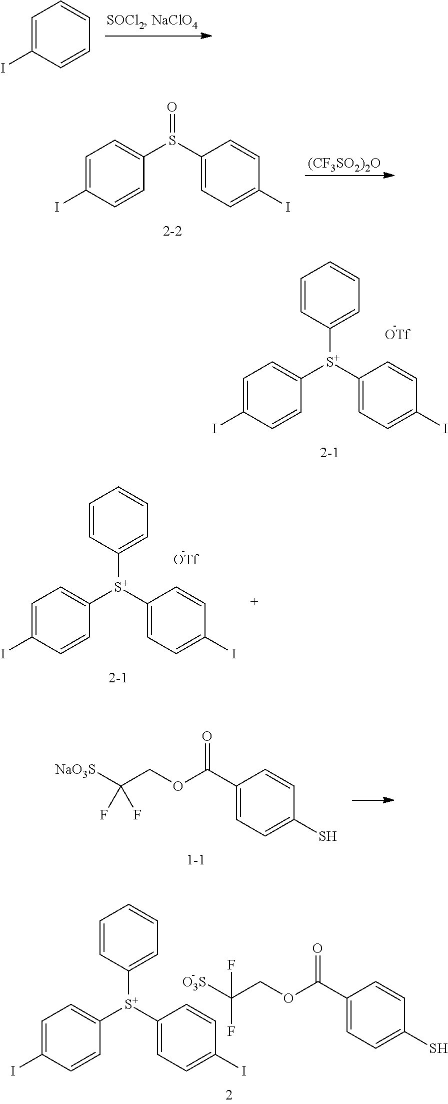

### (1) Synthesis of Intermediate 2-1

Intermediate 2-1 was prepared by referring to Korean Patent Publication No. 10-2022-0074627.

4-iodobenzene (2.246 g, 11.01 mmol), thionyl chloride (0.655 g, 5.51 mmol), and sodium perchlorate (0.117 g, 1.10 mmol) were added to 12 mL of tetrahydrofuran (THF) and stirred for 3 hours. After the reaction, the solvent was removed under reduced pressure by distillation. The residue was extracted with 30 mL of water and 30 mL of dichloromethane. The obtained organic layer was dried over Na₂SO₄ and filtered. The filtrate was concentrated under reduced pressure, and the residue was purified by column chromatography to afford Intermediate 2-2, 4,4′-sulfinylbis(iodobenzene). The obtained compound was confirmed using ¹H-NMR.

¹H-NMR (500 MHz, CDCl₃): δ 7.05 (d, 4H), 7.42 (d, 4H), LC-MS m/z=454.85 (M₊H)

Intermediate 2-2 (3.73 g, 8.20 mmol) was dissolved in 15 mL of benzene, and trifluoromethanesulfonic anhydride (2.778 g, 9.85 mmol) was added dropwise at 0° C. The reaction mixture was stirred at room temperature for 1 hour. After the reaction, the mixture was extracted with 20 mL of water and 50 mL of ethyl acetate. The organic layer was washed with a saturated NaHCO₃ aqueous solution, dried over MgSO₄, and filtered. The filtrate was concentrated under reduced pressure, and the residue was purified by silica gel column chromatography to afford Intermediate 2-1 (4.92 g, 90%). The obtained compound was confirmed using ¹H-NMR and LC-MS.

¹H-NMR (500 MHz, CD₂Cl₂): δ 8.08 (d, 4H), 7.84 (t, 1H), 7.74 (t, 2H), 7.69 (d, 2H), 7.41 (d, 4H),

LC-MS m/z=514.88 (Cation)

### (2) Synthesis of Organic Salt 2

Using the same synthesis method as in Synthesis Example 1 for Organic Salt 1, except that Intermediate 2-1 was used instead of triphenylsulfonium triflate, Organic Salt 2 was obtained with a yield of 64.3%. The obtained compound was confirmed using ¹H-NMR and MALDI.

¹H-NMR (500 MHz, CD₂Cl₂): δ 8.08 (d, 4H), 7.89 (d, 2H), 7.84 (t, 1H), 7.74 (t, 2H), 7.69 (d, 2H), 7.50 (d, 2H), 7.41 (d, 4H), 4.63 (t, 2H), 3.34 (s, 1H),

HRMS (MALDI) calculated for C₂_{₇}H₂_{₀}F₂I₂O₅S₃: m/z 811.85, found: 811.85.

### Synthesis Example 3: Synthesis of Organic Salt 3

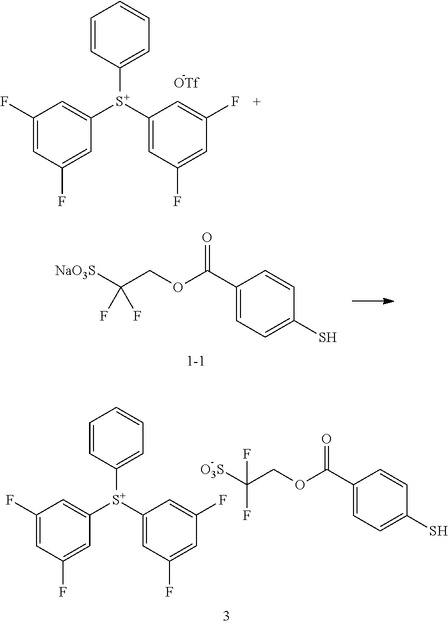

Using the same synthesis method as in Synthesis Example 1 for Organic Salt 1, except that bis(3,5-difluorophenyl)(phenyl)sulfonium triflate was used instead of triphenylsulfonium triflate, Organic Salt 3 was obtained with a yield of 72.7%. The obtained compound was confirmed using ¹H-NMR and MALDI.

¹H-NMR (500 MHz, CD₂Cl₂): δ 7.89 (m, 3H), 7.80 (m, 4H), 7.50 (d, 2H), 7.38 (m, 4H), 7.30 (m, 2H), 4.63 (t, 2H), 3.34 (s, 1H),

HRMS(MALDI) calcd for C27H18F6O5S3: m/z 632.02 Found: 632.01.

### Synthesis Example 4: Synthesis of Organic Salt 4

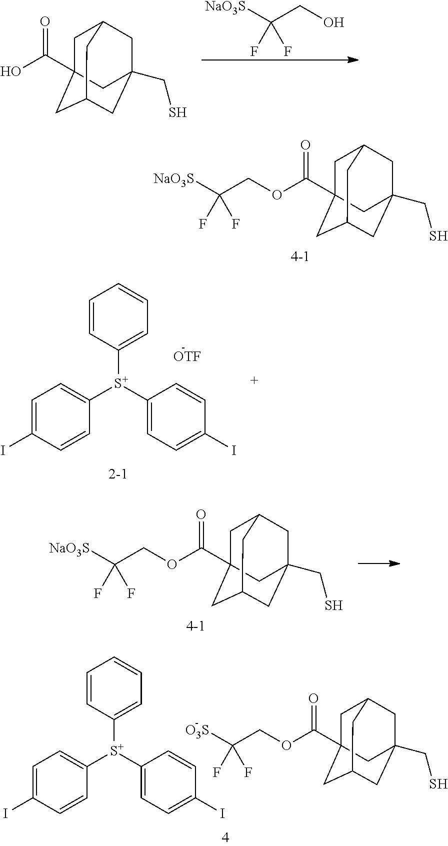

### (1) Synthesis of Intermediate 4-1

Using the same synthesis method as in Synthesis Example 1 for Intermediate 1-1, except that 3-(mercaptomethyl)adamantane-1-carboxylic acid was used instead of 4-mercaptobenzoic acid, Intermediate 4-1 was obtained with a yield of 73.2%. The obtained compound was confirmed using ¹H-NMR and LC-MS.

¹H-NMR (500 MHz, CD₂Cl₂): δ 4.63 (t, 2H), 2.35 (d, 2H), 2.15 (m, 2H), 1.89 (m, 2H), 1.81 (m, 2H), 1.60 (m, 8H), 1.14 (t, 1H),

LC-MS m/z=369.06 (anion).

### (2) Synthesis of Organic Salt 4

Using the same synthesis method as in Synthesis Example 2 for Organic Salt 2, except that Intermediate 4-1 was used instead of Intermediate 1-1, organic salt 4 was obtained with a yield of 66.5%. The obtained compound was confirmed using ¹H-NMR and MALDI.

¹H-NMR (500 MHz, CD₂Cl₂): δ 8.08 (d, 4H), 7.84 (t, 1H), 7.74 (t, 2H), 7.69 (d, 2H), 7.41 (d, 4H), 4.63 (t, 2H), 2.35 (d, 2H), 2.15 (m, 2H), 1.89 (m, 2H), 1.81 (m, 2H), 1.60 (m, 8H), 1.14 (t, 1H),

HRMS (MALDI) calculated for C32H32F2I₂O₅S₃: m/z 833.95, found: 833.94.

### Synthesis Example 5: Synthesis of Organic Salt 5

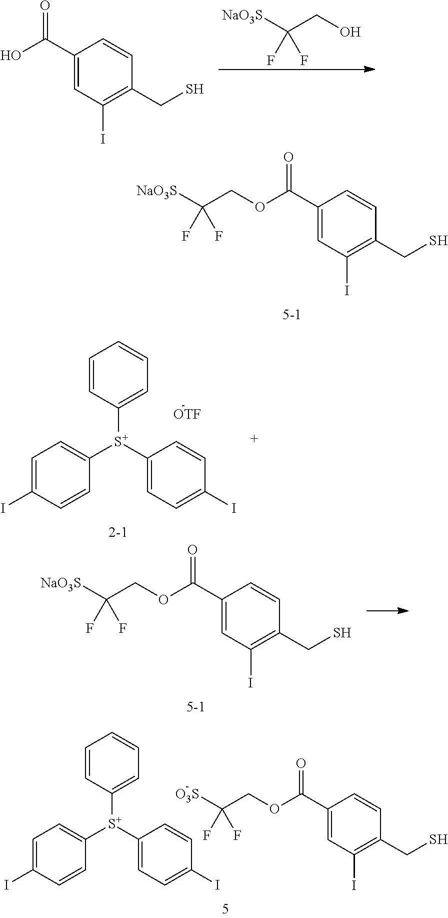

### (1) Synthesis of Intermediate 5-1

Using the same synthesis method as in Synthesis Example 1 for Intermediate 1-1, except that 3-iodo-4-(mercaptomethyl)benzoic acid was used instead of 4-mercaptobenzoic acid, Intermediate 5-1 was obtained with a yield of 52.8%. The obtained compound was confirmed using ¹H-NMR and LC-MS.

¹H-NMR (500 MHz, CD₂Cl₂): δ 8.33 (d, 1H), 7.89 (d, 1H), 7.58 (d, 1H), 4.63 (t, 2H), 1.42 (t, 1H),

LC-MS m/z=369.06 (anion).

### (2) Synthesis of Organic Salt 5

Using the same synthesis method as in Synthesis Example 2 for Organic Salt 2, except that Intermediate 5-1 was used instead of Intermediate 1-1, organic salt 5 was obtained with a yield of 62.4%. The obtained compound was confirmed using ¹H-NMR and MALDI.

¹H-NMR (500 MHz, CD₂Cl₂): δ 8.33 (d, 1H), 8.08 (d, 4H), 7.89 (d, 1H), 7.84 (t, 1H), 7.74 (t, 2H), 7.69 (d, 2H), 7.58 (d, 1H), 7.41 (d, 4H), 4.63 (t, 2H), 1.42 (t, 1H),

HRMS (MALDI) calculated for C₂_{₈}H₂₁F₂I₃O₅S₃: m/z 951.77, found: 951.76.

### Synthesis Example 6: Synthesis of Polymer HS/EAd

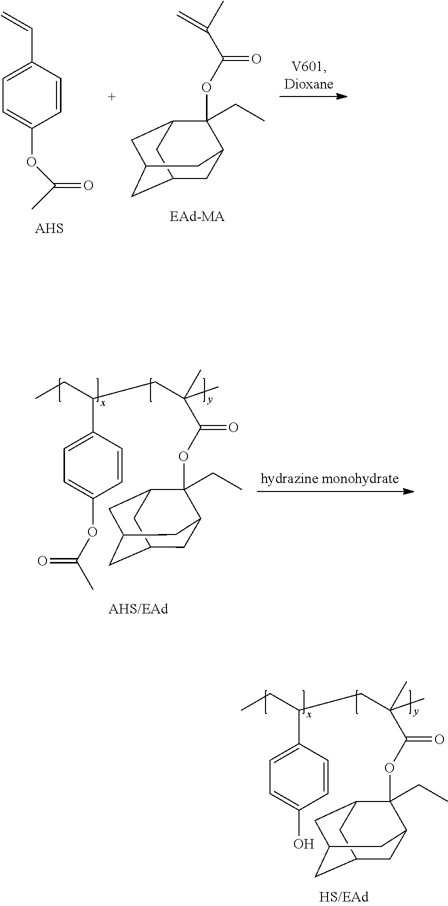

1.5 g (9.3 mmol) of acetoxystyrene (AHS), 2.3 g (9.3 mmol) of 2-ethyl-2-adamantyl methacrylate (EAd-MA), and 0.2 g (0.9 mmol) of azo initiator (V601) were dissolved in 18 mL of dioxane and reacted at 80° C. for 4 hours to obtain AHS/EAd. To the obtained AHS/EAd, 1 g of hydrazine monohydrate was added, and the reaction was carried out at room temperature for 2 hours. Then, 50 mL of deionized water (DW) and 2 g of acetic acid were added to the reaction mixture, which was extracted with ethyl acetate (EA). The obtained solution was precipitated in hexane, and the resulting precipitate was dried at 40° C. for 24 hours, yielding a white powder of polymer HS/EAd. The number-average molecular weight (Mn) of the obtained polymer HS/EAd was 5000, and the polydispersity index (PDI) was 1.3. The molar ratio of HS to EAd in the obtained polymer HS/EAd was 50:50.

### Evaluation Example 1: Evaluation of Acid Generation Effect

Cyclohexanone was used as a solvent, and 10 wt % of poly(4-vinylphenol) (Mw is about 11,000, supplier: Sigma-Aldrich), 5 wt % of Coumarin 6 (CAS No. 38215-36-0), and 5 wt % of a photoacid generator (PAG) were added. Then, additives were further introduced at the contents specified in Table 1 below. The resulting solution was applied onto a 1-inch×1-inch quartz plate by spin-coating to form a 400 nm-thick film, followed by drying at 130° C. for 2 minutes. The film was exposed to DUV (248 nm) at 10 mJ/cm², and the absorbance was measured. Theoretically, Coumarin 6 absorbs the light at 460 nm, but when exposed to acid, its absorbance increases at 535 nm, making Coumarin 6 a suitable acid indicator. After exposure to 10 mJ/cm², the absorbance intensity was normalized based on the assumption that Coumarin 6 was 100% converted. The acid generation level at each exposure dose was expressed as a relative value based on the reference value of Comparative Example 1-1.

TABLE 1

| | | | Additive | Acid generation |
|---|---|---|---|---|
| | Photoacid | | content | level @ DUV |
| | generator | Additives | (wt %) | 10 mJ/cm² |
| Example 1-1 | PAG A | 1 | 0.1 | 1.36 |
| Example 1-2 | PAG A | 1 | 0.5 | 2.95 |
| Example 1-3 | PAG A | 2 | 0.1 | 1.42 |
| Example 1-4 | PAG A | 2 | 0.5 | 3.24 |
| Example 1-5 | PAG A | 3 | 0.1 | 1.54 |
| Example 1-6 | PAG A | 3 | 0.5 | 3.47 |
| Example 1-7 | PAG A | 4 | 0.5 | 4.48 |
| Example 1-8 | PAG A | 5 | 0.5 | 4.63 |
| Comparative | PAG A | — | — | 1.00 |
| Example 1-1 | | | | |
| Comparative | PAG B | — | — | 1.04 |
| Example 1-2 | | | | |

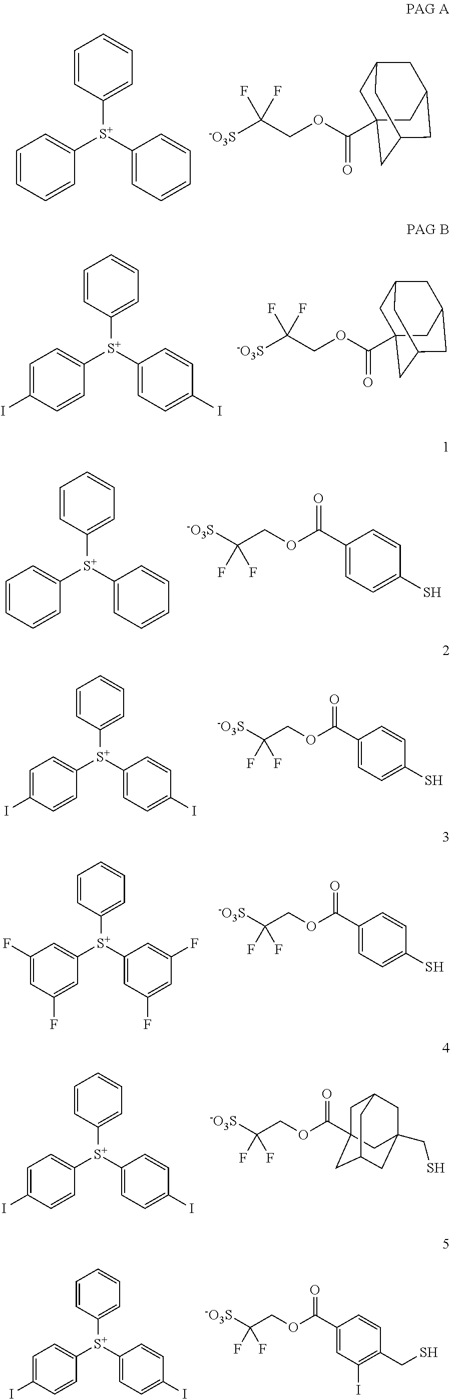

Referring to Table 1, it may be observed that Examples 1-1 to 1-8 exhibit an improved acid generation effect compared to Comparative Examples 1-1 to 1-2. In other words, when using additives containing —SH groups, as in Examples 1-1 to 1-8, it is expected that an enhanced resolution may be achieved even with the same amount of photoacid generator.

### Evaluation Example 2: Evaluation of Acid Diffusion Length (ADL)

The ADL evaluation was conducted by referring to the method disclosed in Macromolecules, 43(9), 4275 (2010).

First, a 12-inch circular silicon wafer substrate was pretreated in a UV ozone cleaning system for 10 minutes. A 1.6 wt % solution of HS/EAd in a propylene glycol methyl ether/propylene glycol methyl ether acetate (PGME/PGMEA) 7/3 (w/w) mixture was applied onto the silicon wafer substrate by spin-coating at 1500 rpm for 30 seconds, forming a 100 nm-thick first film.

Separately, HS/EAd was dissolved in a 1.6 wt % PGME/PGMEA 7/3 (w/w) solution and applied onto a hydrophilized PDMS surface by spin-coating (treated using a UVO cleaner) to form a 100 nm-thick layer. A photoacid generator (PAG) was then added at 50 wt % relative to HS/EAd. Next, an additive listed in Table 2 was introduced at 5 wt % relative to HS/EAd, and the resulting mixture was applied by spin-coating at 1500 rpm for 30 seconds. A second film was then formed by exposing the layer to deep UV (DUV) at 248 nm (100 to 200 mJ/cm²). Due to the exposure, acid was generated from the PAG in the second film.

The second film was then transferred onto the first film by placing it in contact with the first film, applying pressure, and removing PDMS, resulting in a laminated structure consisting of the silicon wafer substrate, the first film, and the second film. The laminated structure was maintained at 90° C. for 60 seconds, allowing the acid generated in the second film to diffuse into the first film. Finally, the laminated structure was washed with a 2.38 wt % tetramethylammonium hydroxide (TMAH) aqueous solution, and the remaining thickness of the first film was measured to evaluate ADL. The ADL values were expressed as relative values, using Comparative Example 2-1 as the reference.

TABLE 2

| | | | Additive | |
|---|---|---|---|---|
| | Photoacid | | content | Opponent |
| | generator | Additives | (wt %) | ADL |
| Example 2-1 | PAG A | 1 | 5 | 0.74 |
| Example 2-2 | PAG A | 2 | 5 | 0.70 |
| Example 2-3 | PAG A | 3 | 5 | 0.72 |
| Example 2-4 | PAG A | 4 | 5 | 0.61 |
| Example 2-5 | PAG A | 5 | 5 | 0.59 |
| Comparative | PAG A | — | — | 1.00 |
| Example 2-1 | | | | |
| Comparative | PAG B | — | — | 0.88 |
| Example 2-2 | | | | |

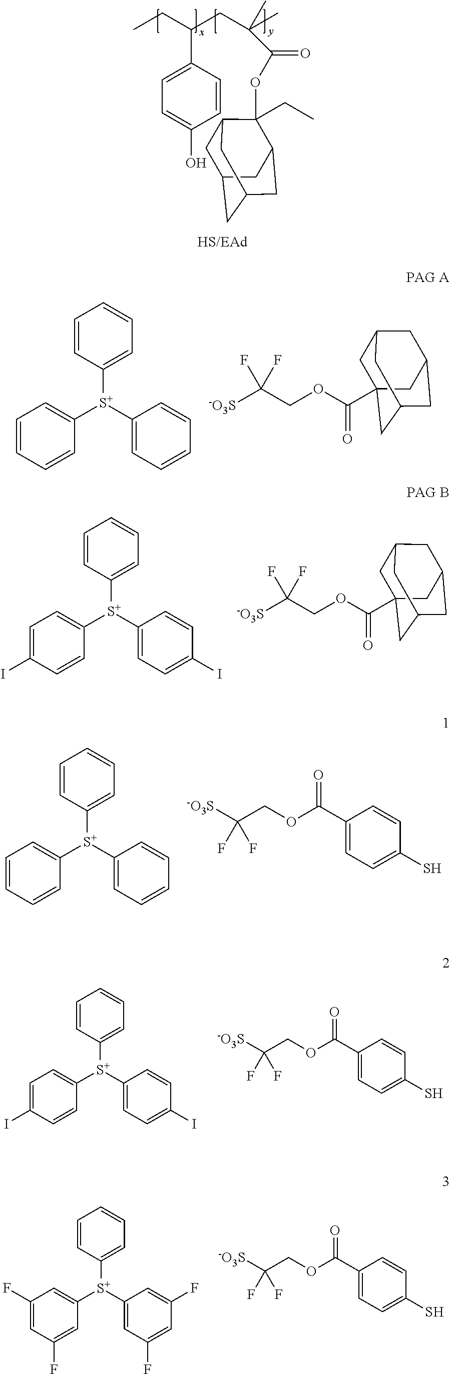

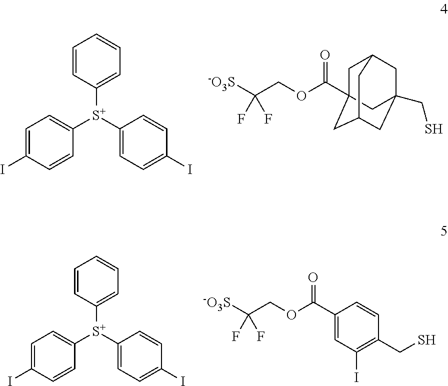

Referring to Table 2, it was observed that Examples 2-1 to 2-5 exhibited significantly reduced ADL values compared to Comparative Examples 2-1 and 2-2. This suggests that, when acid generated by exposure diffuses, the additives in Examples 2-1 to 2-5 more effectively suppressed acid diffusion compared to Comparative Examples 2-1 and 2-2, leading to a more uniform acid confinement.

### Evaluation Example 3: Thin Film Phenomena Evaluation (EUV)

A casting solution was prepared by dissolving polymer HS/EAd at 1.6 wt % in a propylene glycol methyl ether/propylene glycol methyl ether acetate (PGME/PGMEA) 7/3 (w/w) solvent mixture. Then, 0.024 mmol of PAG and 0.016 mmol of PDQ were added. Next, additives were introduced as listed in Table 3, and the mixture was filtered through a 0.2 μm membrane filter to prepare the final casting solution. A 12-inch diameter silicon wafer was 02 plasma-treated for 30 minutes, followed by spin-coating the casting solution at 1500 rpm for 1 minute. The coated film was then pre-baked (PAB) at 110° C. for 1 minute, resulting in an initial film thickness of approximately 20.0 nm. A mask (4 cm×4 cm) with a rectangular hole (1 cm×1 cm) and 1 cm thick was placed on top of the film. Each hole was exposed to EUV (wavelength 13.5 nm) at doses ranging from 0 to 50 mJ/cm², followed by post-exposure baking (PEB) at 90° C. for 60 seconds. After PEB, the film was developed using a 2.38 wt % TMAH aqueous solution as the developer by immersing it at 25° C. for 20 seconds, followed by rinsing with deionized (DI) water for 10 seconds to remove the exposed regions and drying to form the resist pattern. The resulting photoresist pattern was analyzed using CD-SEM (Critical Dimension Measurement Scanning Electron Microscope) to measure the Eₒₚ, resolution, island pattern uniformity (IPU), and sensitivity. The Eₒₚ values were expressed as relative values, with Comparative Example 3-1 used as the reference, as shown in Table 3.

The measured values of resolution, IPU, and sensitivity were substituted into Equation 1 to calculate the Z-factor, and the results were expressed as relative values based on Comparative Example 3-1, as shown in Table 3.

Z-factor=(resolution)³×(IPU)²×(sensitivity) Equation 1

In Equation 1: Resolution refers to the critical dimension (CD) size (half-pitch). IPU (Island Pattern Uniformity) is a value calculated from the CD variation. Sensitivity corresponds to Eₒₚ. A lower Z-factor indicates better pattern performance at the same exposure dose.

TABLE 3

| | | Additive content | Z-factor | Eₒₚ |
|---|---|---|---|---|
| | | (wt % compared | (relative | (relative |
| | Additives | to HS/EAd) | value) | value) |
| Example 3-1 | 2 | 1 | 90.6 | 0.82 |
| Example 3-2 | 2 | 5 | 94.8 | 0.53 |
| Example 3-3 | 4 | 1 | 93.3 | 0.91 |
| Example 3-4 | 4 | 5 | 98.5 | 0.74 |
| Example 3-5 | 5 | 1 | 92.6 | 0.87 |
| Example 3-6 | 5 | 5 | 97.4 | 0.65 |
| Comparative | — | — | 100 | 1 |
| Example 3-1 | | | | |

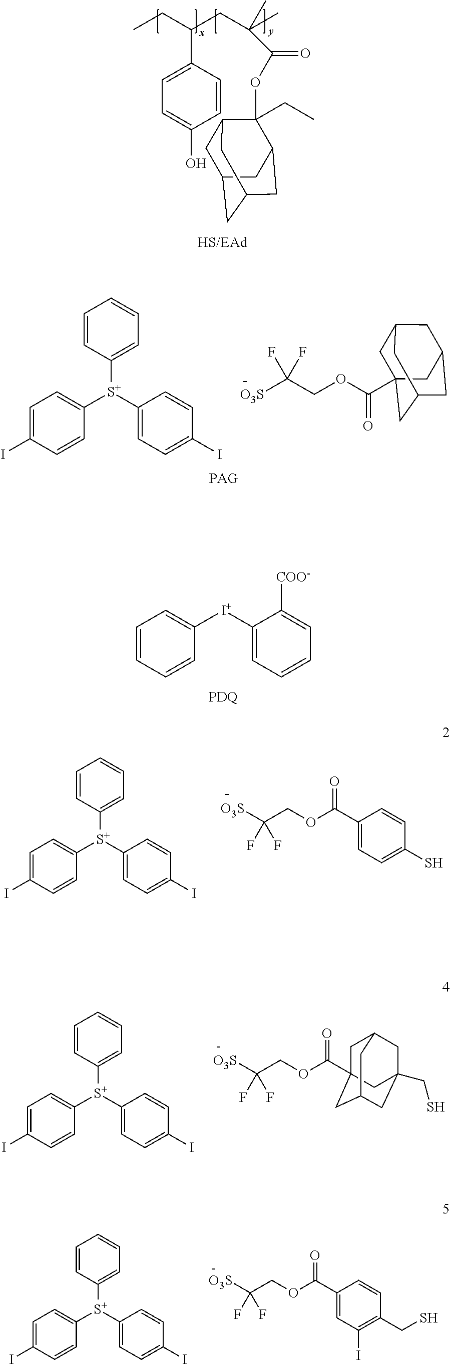

Referring to Table 3 above, it was confirmed that the patterns of Examples 3-1 to 3-6 exhibited lower Z-factor and Eₒₚ than the pattern of Comparative Example 3-1.

The embodiments of the disclosure may provide a resist composition with enhanced sensitivity, improved resolution, and/or reduced defects.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope as defined by the following claims.

What is claimed is:

1. An organic salt represented by Formula 1: 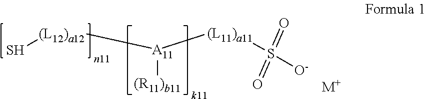
wherein, in Formula 1,
A₁₁ is a linear or cyclic C₁-C₃₀ hydrocarbon group optionally including a heteroatom,
k11 is selected from integers of 1 to 4,
L₁₁ and L₁₂ are each independently: a single bond; O; S; C(═O); C(═O)O; OC(═O); C(═O)NR₁₂; NR₁₂C(═O); S(═O); S(═O)₂; S(═O)₂O; OS(═O)₂; or a linear, branched or cyclic C₁-C₃₀ divalent hydrocarbon group optionally including a heteroatom,
a11 and a12 are each independently selected from integers of 0 to 4,
R₁₁ and R₁₂ are each independently hydrogen, deuterium, a halogen atom, a cyano group, a hydroxyl group, or a linear, branched or cyclic C₁-C₂₀ monovalent hydrocarbon group optionally including a heteroatom,
b11 is selected from integers of 0 to 10,
any two adjacent groups among R₁₁, L₁₁ and L₁₂ are optionally bonded to each other to form a ring,
any two adjacent groups among a plurality of R₁₁ groups are optionally bonded to each other to form a ring,
n11 is selected from integers of 1 to 5, and
M⁺ is a counter cation.

2. The organic salt of claim 1,
wherein A₁₁ is a C₁-C₃₀ alkyl group, a C₂-C₃₀ alkenyl group, a C₃-C₃₀ cycloalkyl group, a C₃-C₃₀ heterocycloalkyl group, a C₃-C₃₀ cycloalkenyl group, a C₃-C₃₀ heterocycloalkenyl group, a C₆-C₃₀ aryl group, or a C₁-C₃₀ heteroaryl group.

3. The organic salt of claim 1,
wherein A₁₁ is a methyl group, an ethyl group, an ethenyl group, a cyclopentyl group, a cyclohexyl group, a tetrahydrofuran group, a tetrahydropyran group, a norbornyl group, a norbornenyl group, a norbornadienyl group, a tricyclodecanyl group, a tetracyclododecanyl group, an adamantyl group, an oxa-norbornyl group, an oxa-tricyclodecanyl group, an oxa-tetracyclododecanyl group, an oxa-adamantyl group, a benzene group, a naphthalene group, a phenanthrene group, an anthracene group, a pyrrole group, a furan group, a thiophene group, an indole group, a benzofuran group, a benzothiophene group, a carbazole group, a dibenzofuran group, or a dibenzothiophene group.

4. The organic salt of claim 1,
wherein L₁₁ and L₁₂ are each independently: a single bond; O; C(═O); C(═O)O; OC(═O); C(═O)NH; NHC(═O); or a substituted or unsubstituted C₁-C₃₀ alkylene group, a substituted or unsubstituted C₃-C₃₀ cycloalkylene group, a substituted or unsubstituted C₃-C₃₀ heterocycloalkylene group, a substituted or unsubstituted C₂-C₃₀ alkenylene group, a substituted or unsubstituted C₃-C₃₀ cycloalkenylene group, a substituted or unsubstituted C₃-C₃₀ heterocycloalkenylene group, a substituted or unsubstituted C₆-C₃₀ arylene group, a substituted or unsubstituted C₁-C₃₀ heteroarylene group, or a combination thereof.

5. The organic salt of claim 1,
wherein R₁₁ and R₁₂ are each independently selected from: hydrogen; deuterium; a halogen atom; a cyano group; a hydroxyl group; and a C₁-C₂₀ alkyl group, a C₁-C₂₀ alkoxy group, a C₃-C₂₀ cycloalkyl group, a C₃-C₂₀ cycloalkoxy group, and a C₆-C₂₀ aryl group, each unsubstituted or substituted with deuterium, a halogen atom, a cyano group, a hydroxyl group, an amino group, a carboxylate group, an ester moiety, a sulfonate moiety, a carbonate moiety, a lactone moiety, a sultone moiety, a carboxylic anhydride moiety, a C₁-C₂₀ alkyl group, a C₁-C₂₀ halogenated alkyl group, a C₁-C₂₀ alkoxy group, a C₃-C₂₀ cycloalkyl group, a C₃-C₂₀ cycloalkoxy group, a C₆-C₂₀ aryl group, or a combination thereof.

6. The organic salt of claim 1,
n11 is 1 or 2.

7. The organic salt of claim 1,
wherein M⁺ is represented by one of Formulae 2-1 and 2-2: 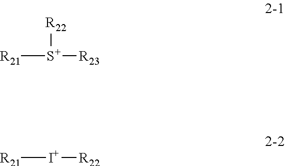
wherein in Formulae 2-1 and 2-2,
R₂₁ to R₂₃ are each independently a linear, branched or cyclic C₁-C₃₀ monovalent hydrocarbon group optionally including a heteroatom, and
any two adjacent groups of R₂₁ to R₂₃ are optionally bonded to each other to form a ring.

8. The organic salt of claim 7,
wherein R₂₁ to R₂₃ are each independently selected from a C₁-C₂₀ alkyl group, a C₃-C₂₀ cycloalkyl group, and a C₆-C₂₀ aryl group, each unsubstituted or substituted with deuterium, a halogen atom, a cyano group, a hydroxyl group, a carboxyl group, an ester moiety, a sulfonate moiety, a carbonate moiety, a lactone moiety, a sultone moiety, a carboxylic anhydride moiety, a C₁-C₂₀ alkyl group, a C₁-C₃₀ halogenated alkyl group, a C₁-C₂₀ alkoxy group, a C₃-C₂₀ cycloalkyl group, a C₃-C₂₀ cycloalkoxy group, a C₆-C₂₀ aryl group, or a combination thereof.

9. The organic salt of claim 1,
wherein M⁺ is represented by one of Formulae 2-11 and 2-12: 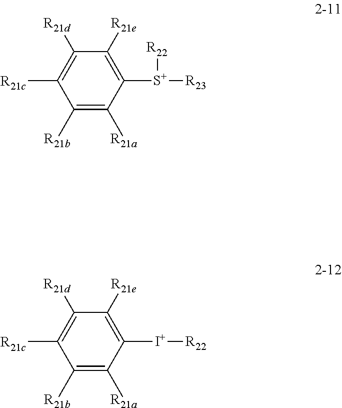
wherein in Formulae 2-11 and 2-12,
R₂₁ₐ to R₂₁ₑ are each independently: hydrogen; deuterium; a halogen atom; a cyano group; a hydroxyl group; or a linear, branched or cyclic C₁-C₂₀ monovalent hydrocarbon group optionally including a heteroatom,
R₂₂ to R₂₃ are each independently a linear, branched or cyclic C₁-C₃₀ monovalent hydrocarbon group optionally including a heteroatom,
any two adjacent groups of R₂₁a to R₂₁e and R₂₂ to R₂₃ are optionally bonded to each other to form a ring.

10. The organic salt of claim 1,
wherein M⁺ is represented by any one of Formulae 2-21 to 2-23: 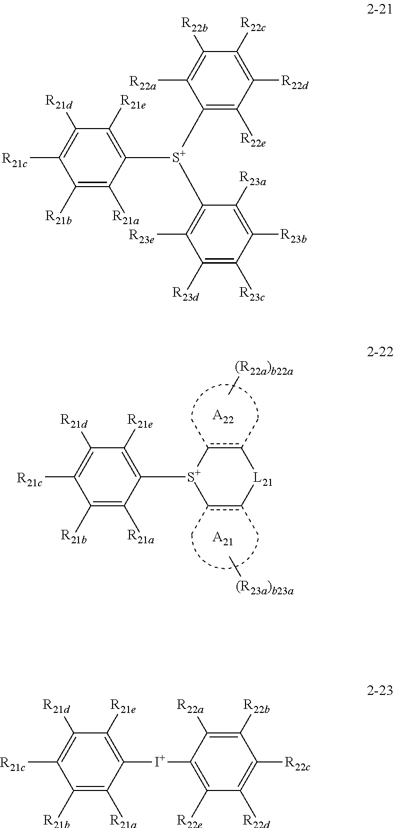
wherein in Formulae 2-21 to 2-23,
R₂₁ₐ to R₂₁ₑ, R₂₂ₐ to R₂₂ₑ, and R₂₃ₐ to R₂₃ₑ are each independently: hydrogen; deuterium; a halogen atom; a cyano group; a hydroxyl group; or a linear, branched or cyclic C₁-C₂₀ monovalent hydrocarbon group optionally including a heteroatom,
any two adjacent groups of R₂₁ₐ to R₂₁ₑ, R₂₂ₐ to R₂₂ₑ, and R₂₃ₐ to R₂₃ₑ are optionally bonded with each other to form a condensed ring,
b22a and b23a are each an integer from 1 to 4,
A₂₁ and A₂₂ are each independently absent or a benzene ring;
each [symbol] is a carbon-carbon single bond or a carbon-carbon double bond;
L₂₁ is a single bond, O, S, CO, SO, SO₂, CRR′, or NR, and
R and R′ are each independently: hydrogen; deuterium; a halogen atom; a cyano group; a hydroxyl group; or a linear, branched or cyclic C₁-C₂₀ monovalent hydrocarbon group optionally including a heteroatom.

11. The organic salt of claim 1,
wherein M⁺ comprises at least one halogen atom.

12. The organic salt of claim 1,
wherein M⁺ is selected from Group 1:

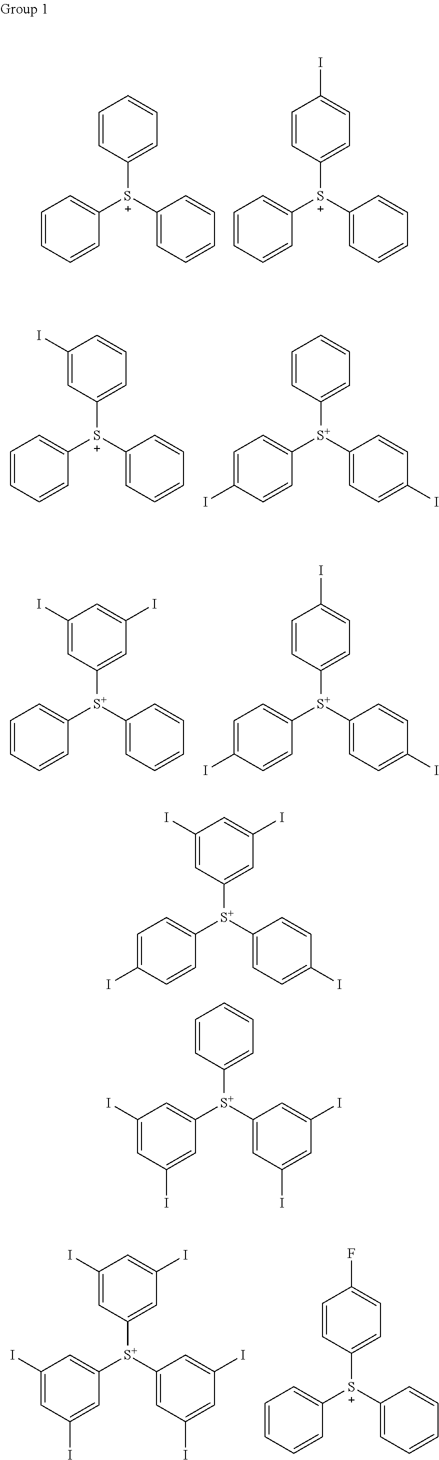

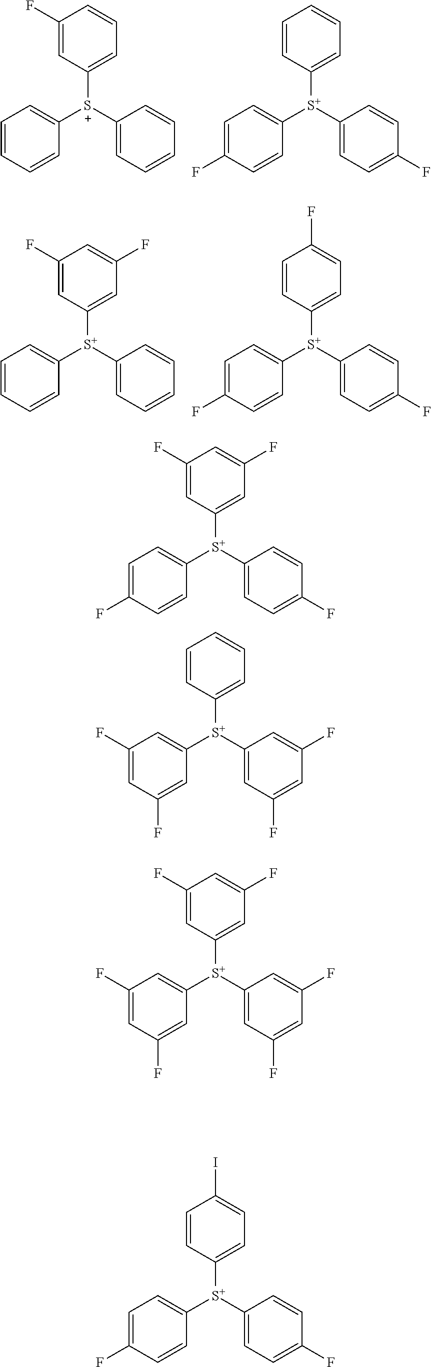

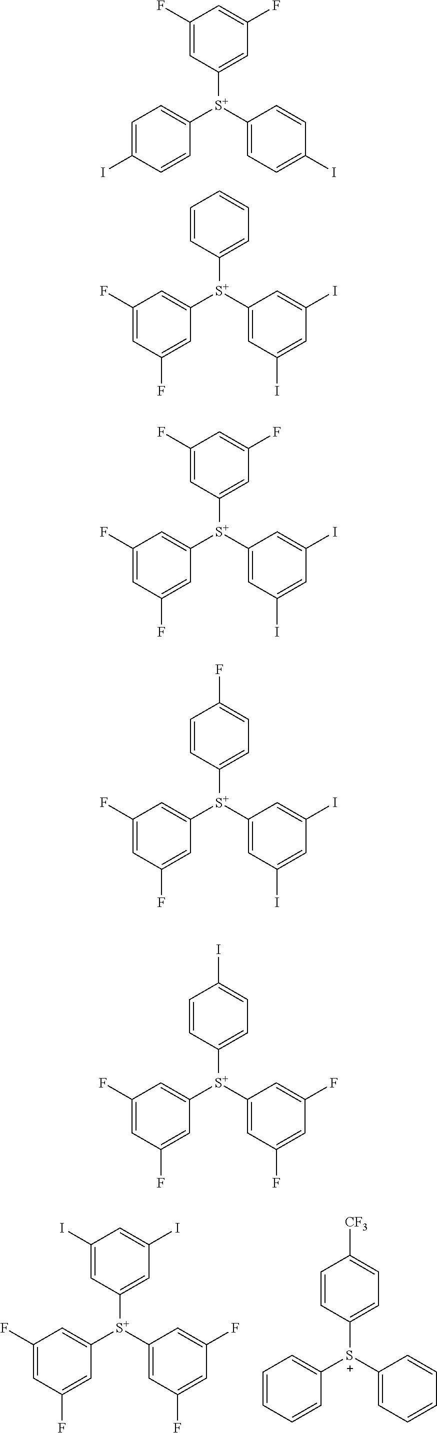

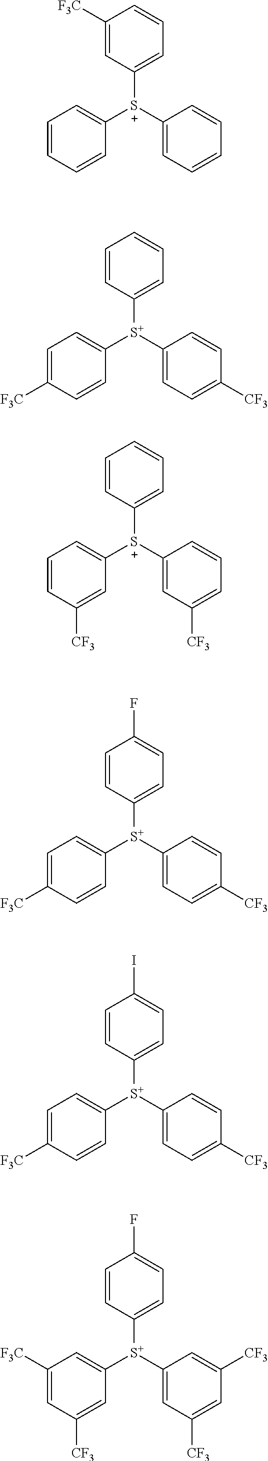 [SMILES: FC(F)(F)c1cccc([S+](c2ccccc2)c2ccccc2)c1 ; FC(F)(F)c1ccc([S+](c2ccccc2)c2ccc(C(F)(F)F)cc2)cc1 ; FC(F)(F)c1cccc([S+](c2ccccc2)c2cccc(C(F)(F)F)c2)c1 ; Fc1ccc([S+](c2ccc(C(F)(F)F)cc2)c2ccc(C(F)(F)F)cc2)cc1 ; FC(F)(F)c1ccc([S+](c2ccc(I)cc2)c2ccc(C(F)(F)F)cc2)cc1 ; Fc1ccc([S+](c2cc(C(F)(F)F)cc(C(F)(F)F)c2)c2cc(C(F)(F)F)cc(C(F)(F)F)c2)cc1]

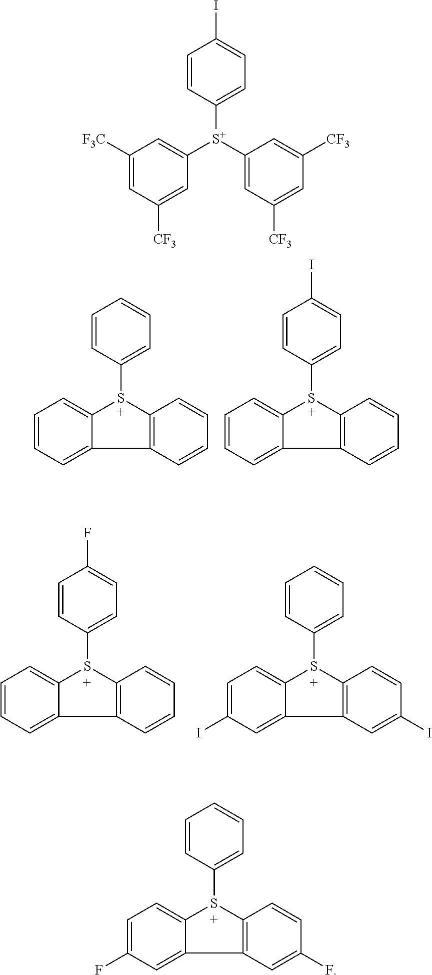

13. The organic salt of claim 1,
wherein the organic salt represented by Formula 1 is an organic salt selected from Group II: 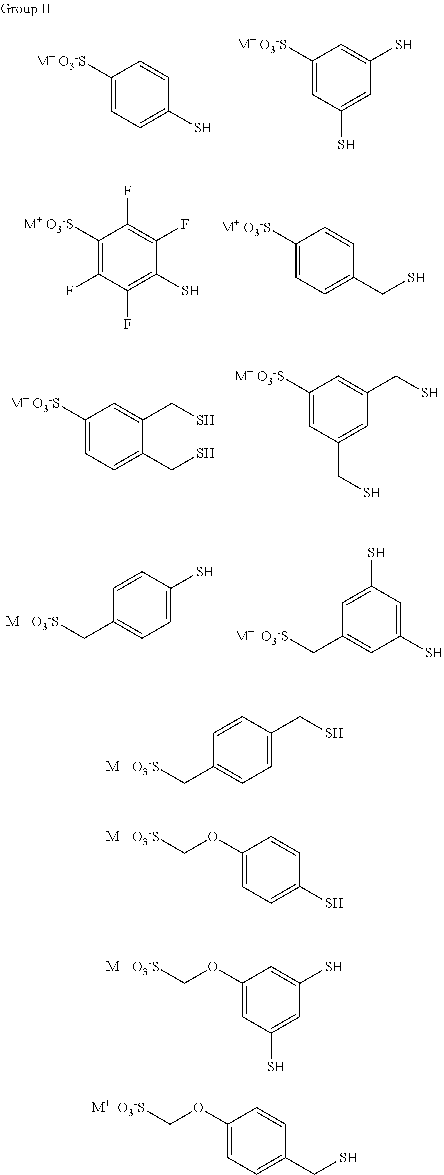 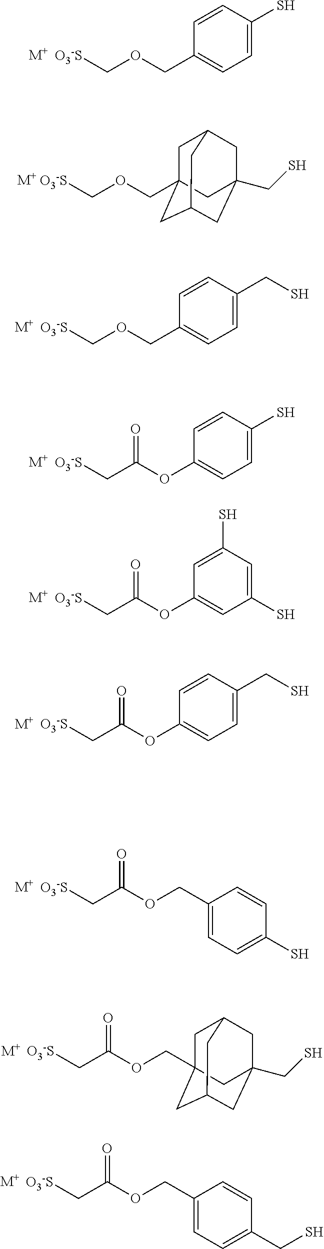 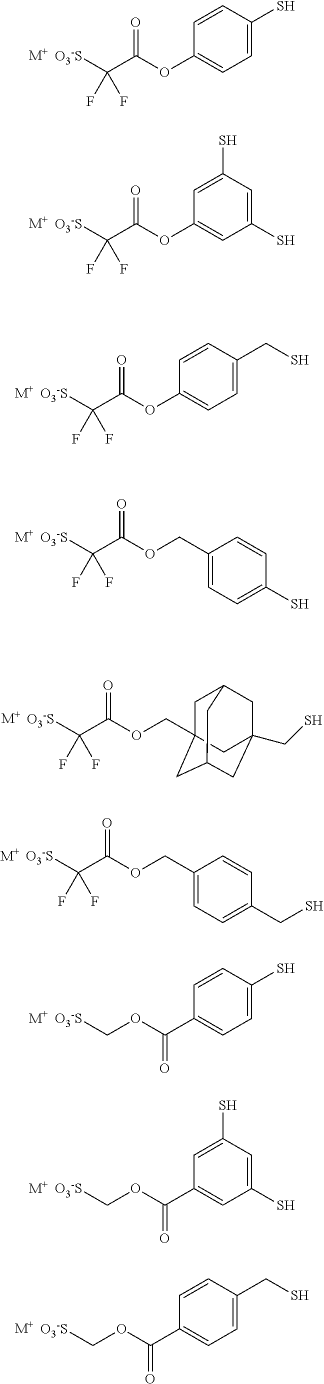 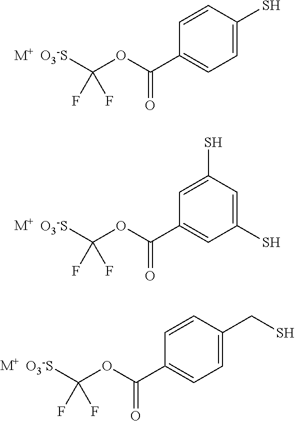 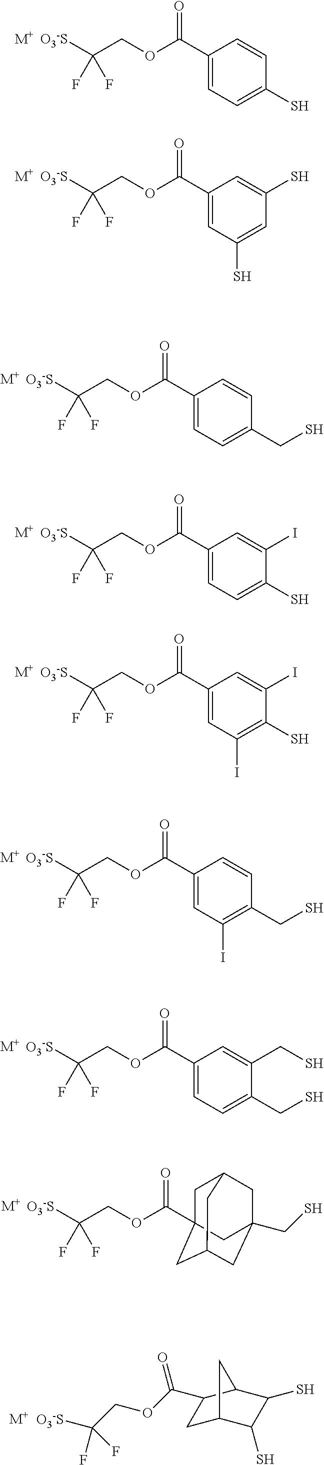 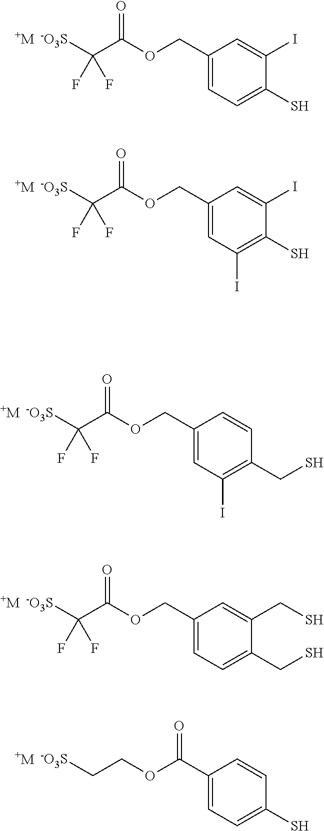
wherein in Group II, M⁺ is a counter cation.

14. A resist composition comprising:
the organic salt of claim 1; a photoacid generator; and
a base resin.

15. The resist composition of claim 14,
wherein the organic salt is included in an amount of about 0.1 parts to about 50 parts by weight, based on 100 parts by weight of the base resin.

16. The resist composition of claim 14,
wherein the organic salt is included in an amount of about 0.01 parts to about 70 parts by weight, based on 100 parts by weight of the photoacid generator.

17. The resist composition of claim 14,
wherein the base resin comprises a first repeating unit represented by Formula 3: 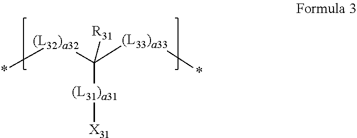
wherein in Formula 3,
L₃₁ to L₃₃ are each independently: a single bond; O; S; C(═O); C(═O)O; OC(═O); C(═O)NR₃₂; NR₃₂C(═O); S(═O); S(═O)₂O; OS(═O)₂; or a linear, branched or cyclic C₁-C₃₀ divalent hydrocarbon group optionally including a heteroatom,
a31 to a33 are each independently an integer from 1 to 4,
R₃₁ and R₃₂ are each independently: hydrogen; deuterium; a halogen atom; a cyano group; a hydroxyl group; an amino group; a carboxylate group; a thiol group; an ester moiety; a sulfonate moiety; a carbonate moiety; a lactone moiety; a sultone moiety;
a carboxylic anhydride moiety; or a linear, branched or cyclic C₁-C₃₀ monovalent hydrocarbon group optionally including a heteroatom,
X₃₁ is an acid labile group, and
* is a bonding site with a neighboring atom.

18. A method of forming a pattern, comprising:
applying the resist composition of claim 14 onto a substrate, to form a resist film;
exposing at least a portion of the resist film with high-energy rays to provide an exposed resist film; and
developing the exposed resist film by using a developer.

19. The method of claim 18,
wherein the exposing is performed by irradiating at least one of ultraviolet rays, deep ultraviolet (DUV) rays, extreme ultraviolet (EUV) rays, X-rays, γ-rays, electron beams (EBs), or a particle beams.

20. The method of claim 18,
wherein the exposed resist film includes an exposed portion and a non-exposed portion, and
wherein the exposed portion is removed in the developing.
